(19) United States
(12) Patent Application Publication
(10) Pub. No.: US 2011/0003022 A1
(43) Pub. Date: Jan. 6, 2011

(54) Composition and Method

(76) Inventors: Mark Stephen Baird, Gwynedd (GB); David Preskett, Gwynedd (GB)

Correspondence Address: HOFFMAN WARNICK LLC, 75 STATE STREET, 14TH FLOOR, ALBANY, NY, 12207, US

(21) Appl. No.: 12/810,010

(22) PCT Filed: Dec. 22, 2008
(86) PCT No.: PCT/GB08/51224

§ 371 Date: Sep. 13, 2010

(30) Foreign Application Priority Data
Dec. 21, 2007 (GB) 0724967.5

(51) Int. Cl. A01N 65/00; C07C 33/02; C07C 57/03; C07J 53/00

(52) U.S. Cl. 424/777; 424/725; 568/857; 562/598; 552/514; 536/5; 554/223

(57) ABSTRACT

A process of obtaining a saponin-rich component from a plant of the Araliaceae family, the process comprising the steps of: (a) treating a portion of the plant with an extraction solvent in which saponin-containing compounds are soluble; and (b) treating the portion of plant or the extract obtained therefrom to remove fatty acid residues from said portion of plant or extract thereof.

The present invention relates to compounds obtainable from the Araliaceae family of plants, to compositions comprising the same and to uses therefor.

The Araliaceae family of plants comprises two subfamilies, the Araliodideae and the Hydrocotyloideae subfamilies. The genera of plants covered by the Araliodideae subfamily include Anakasia, Apiopetalum, Aralia, Arthrophyllum, Astrotricha, Boninofatsia, Brassaiopsis, Cephalaralia, Chemodendron, Cromapanax, Cuphocarpus, Cussonia, Dendropanax, Eleutherococcus, ×Fatshedera, Fatsia, Gamblea, Gastonia, Harmsiopanax, Hedera, Heteropanax, Hunaniopanax, Kalopanax, Mackinlaya, Macropanax, Megalopanax, Merrilliopanax, Meryta, Metapanax, Motherwellia, Munroidendron, Oplopanax, Oreopanax, Osmoxylon, Panax, Polyscias, Pseudopanax, Pseudosciadium, Raukaua, Reynoldsia, Schefflera, Sciadodendron, Seemannaralia, Sinopanax, Stilbocarpa, Tetrapanax, Tetraplasandra, Trevesia and Woodburnia.

The subfamily Hydrocotyloideae includes the genera Azorella, Centella, Hydrocotyle, Platysace and Xanthosia.

Of the genera of species in the Araliaceae plant family, the present invention relates in particular to those of the Hedera genus. Species of the Hedera genus include Hedera algeriensis, Hedera azorica, Hedera canariensis, Hedera caucasigena, Hedera colchica, Hedera cypria, Hedera helix, Hedera hibernica, Hedera maderensis, Hedera maroccana, Hedera nepalensis, Hedera pastuchowii, Hedera rhombea, Hedera sinensis and Hedera taurica.

A wide variety of plant extracts are commonly used in numerous medicinal and industrial applications.

One class of useful compounds obtainable from a number of plants are saponins.

Saponins are so named due to their ability to form stable, soap-like foams at low concentrations (Latin sapo=soap); this ability was used as a quantitative assay of saponins and forms the basis of much literature on the subject.

The nomenclature of saponins was reviewed by Hostettmann and Marston in Saponins, p 10-17, in Phillipson, J. D. (ed.) Chemistry and Pharmacology of Natural Products (series), CUP, Cambridge, 1995, starting with two basic skeletons; steroidal, having 27 carbon atoms or triterpenoid, with 30 carbon atoms. The less widely distributed steroidal forms are further divided into two classes, the spirostanes, found principally in monocotyledons such as lilies, onions, yucca and agave and the furostanes. This latter steroidal group are of considerable commercial importance as a platform molecule for the manufacture of steroid hormones, principally from Dioscorea. A recent review has classified dammaranes, lupanes, hopanes, oleananes, ursanes and steroids according to their biosynthetic pathways.

The triterpenes are a larger group consisting of two principal structural classes; either tetracyclic or pentacyclic. Tetracyclic structures are sub-divided as dammaranes, cucurbitanes and lanostanes. The pentacyclic structure forms the largest single group, the oleananes. Other major pentacyclic triterpene classes are ursanes and lupanes while minor classes of taraxeranes, taraxastanes and friedolanes are also recognised.

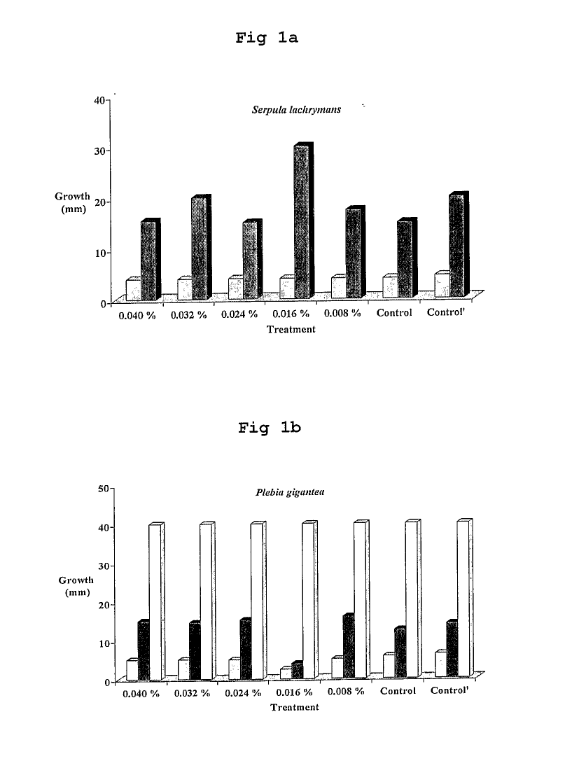

The core skeleton molecules are termed aglycones (also referred to as genins or sapogenins) and are not usually found without substituents attached. The carbon skeleton of the most common aglycones of the oleanane class are shown in FIG. 1.

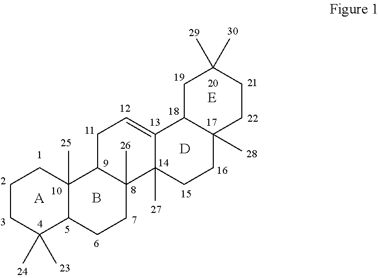

FIG. 1 shows the carbon skeleton of the olean-12-en aglycone structure. Principal attachments are most commonly found at the C3 and C28 positions. Other attachment points are usually linked to an OH group, typically at the C2, C23 or C24 positions.

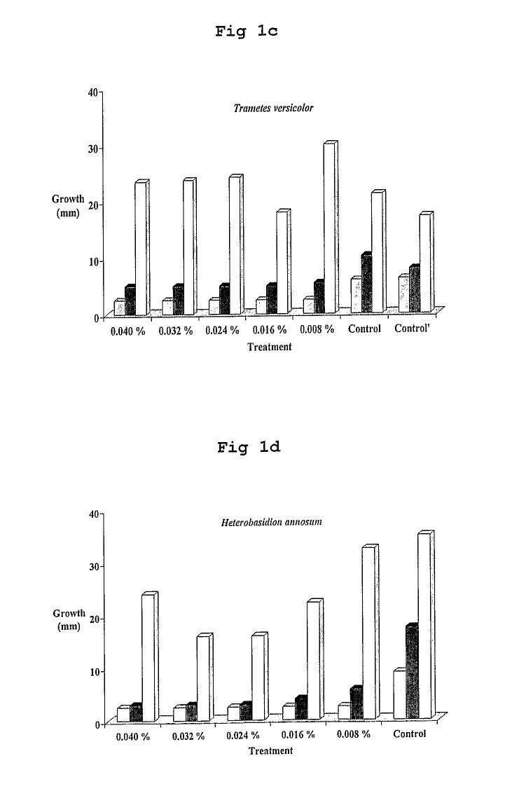

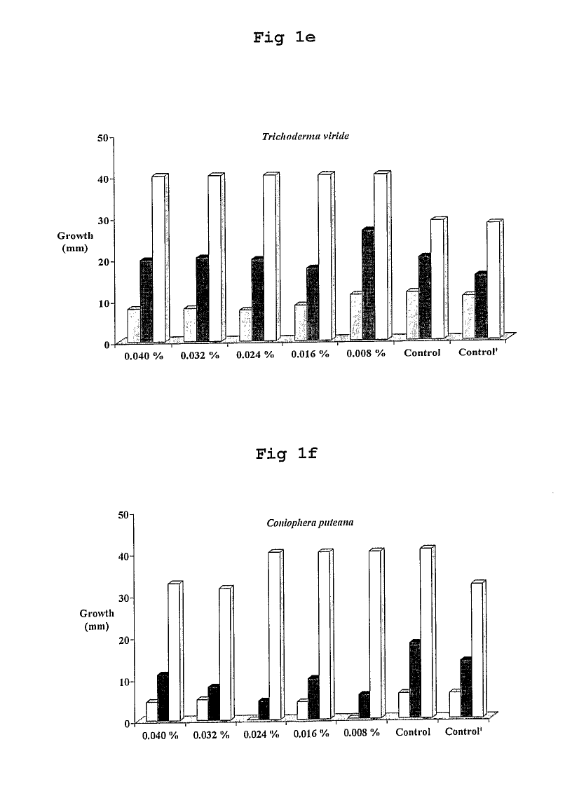

Of the oleananes, which occur in most orders of the plant kingdom, the aglycones most commonly found are oleanolic acid shown in FIG. 2, followed by hederagenin shown in FIG. 3.

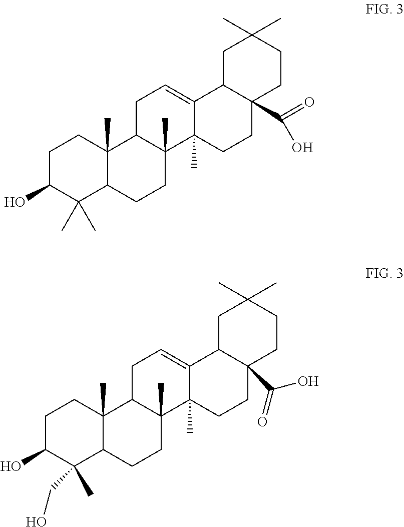

In broad terms, sugars are the principal substituents found generally either as monosaccharides or as polysaccharide chains, although in Calendula officianalis (marigolds), saturated fatty acids are sometimes bound to the aglycone at the C3 position. It will be appreciated however that natural sources of saponins comprise complex mixtures of compounds and that the amounts of different compounds present in a sample will vary considerably from species to species. They also vary within the different parts of the plant.

The present invention relates to the extraction, modification and use of saponin compounds from plants of the Araliaceae family, in particular those obtainable from the Hedera species, for example Hedera helix.

According to a first aspect of the present invention, there is provided a process of obtaining a saponin-rich component from a plant of the Araliaceae family, the process comprising the steps of:

(a) treating a portion of the plant with an extraction solvent in which saponin-containing compounds are soluble; and

(b) treating the portion of plant or the extract obtained therefrom to remove fatty acid residues from said portion of plant or extract thereof.

The plant may be selected from any saponin-containing plant of the Araliaceae family including all of the genera listed above. Preferably the plant is selected from the Hedera genus. Most preferably the plant is Hedera helix.

The inventors have found that a saponin-rich component can be obtained from the leaves, the fruit (including the seed) and other parts of the Hedera helix plant, for example the stems or the bark. However the process provides particularly favourable results when carried out using fruits and/or leaves of the plant. It may also be carried out on only the seeds.

In especially preferred embodiments the portion of plant comprises fruits of the plant. It may comprise the whole fruit, including the fleshy pulp and the seed or it may comprise only the fleshy pulp part of the fruit.

In some embodiments the portion of plant consists essentially of fruit, which may be whole fruit including seeds or only the fleshy portion of the fruit.

The portion of plant may be harvested by any suitable means. It may be harvested by hand or by mechanical means, for example using flails, combined harvesting, by beating or by cutting. Vacuum assisted methods could also be used.

Examples of suitable extraction solvents for use in step (a) include pyridine, THF, DMSO and alcohols.

Preferably the extraction solvent comprises an alcohol. More preferably the extraction solvent comprises an alcohol having 1 to 4 carbon atoms. Most preferably the extraction solvent comprises methanol or ethanol. The extraction solvent may comprise neat methanol and/or ethanol or it may comprise an aqueous solution thereof. For example it may comprise at least 80%, more preferably at least 95% alcohol. In some embodiments the extraction solvent comprises at least 95 wt % ethanol. For example, absolute ethanol or 99 wt % ethanol may be used. In other embodiments, an aqueous alcohol may be used, for example comprising from 50 to 90%, preferably 65 to 85% ethanol by volume.

Preferably the portion of plant is formed into a comminuted form prior to step (a). This may involve taking a sample of the plant and forming it into a paste, for example using a food processor, a pestle and mortar or mincer. Alternatively the plant may be chopped or shredded using a knife or other cutting implement. In some preferred embodiments the plant is processed by hammermilling or grinding into the comminuted form.

In preferred embodiments the portion of plant is dried prior to step (a). This may be before and/or after the plant is formed into a comminuted form. Preferably the portion of plant is processed to provide a comminuted form after drying.

Such a drying step may comprise heating the portion of plant in an oven. Typically this may be for at least an hour, preferably at least four hours, more preferably at least ten hours, for example at least sixteen hours, preferably at least twenty hours. Drying may comprise heating in an oven for up to a week, for example up to three days, for example up to forty hours, for example up to thirty hours.

The drying step may involve heating in an oven at a temperature of at least 35° C., preferably at least 40° C., for example at least 50° C. The drying step may be carried out in an oven having a temperature of up to 250° C., preferably up to 200° C., for example up to 150° C., or up to 120° C. Oven temperatures of 50-60° C. or 80-90° C. may typically be used. Preferably air is circulated over the portion of plant during the drying process.

In some embodiments step (a) may be carried out at ambient temperature.

Preferably however step (a) comprises heating a portion of the plant in the extraction solvent. This may be at a temperature of at least 30° C., preferably at least 35° C., more preferably at least 40° C., for example at least 50° C., preferably at least 60° C. The extraction may be carried out by heating at a temperature of up to 150° C., for example up to 120° C., for example up to 100° C., for example up to 90° C., or up to 80° C. Suitably step (a) comprises heating a portion of plant in a refluxing solvent.

The extraction step (a) is suitably carried out by heating a portion of plant in the extraction solvent for at least 1 hour, for example at least 6 hours, preferably at least 10 hours, more preferably at least 18 hours, for example at least 30 hours.

The plant extract may be heated in the solvent for up to a week, for example up to 5 days, preferably up to 3 days.

Step (a) may comprise heating a portion of the plant in an extraction solvent for more than one period. A further solvent sample may be added and the heating repeated.

Preferably step (a) involves continuous extraction of the saponin compounds. Preferably it is carried out using apparatus which allows percolation of the solvent and soaking of the portion of plant therein. The portion of plant may be suspended loosely in the solvent or held within a removable container.

In some embodiments step (a) may not comprise simply heating the portion of plant in an extraction solvent. If using a supercritical solvent, for example supercritical carbon dioxide, heating may not be necessary. The use of supercritical carbon dioxide as a reaction solvent has a number of advantages, for example it is non-toxic, can be allowed to simply evaporate at the end of a reaction and may allow reactions to be carried out at lower temperatures.

Step (a) may include the use of a microwave or a sonicator with or without heating to assist extraction of saponin-containing compounds into the extraction solvent.

A review paper, Recent advances in extraction of nutraceuticals from plants, Lijun Wang and Curtis L. Weller, Trends in Food Science & Technology, 17 (2006), 300-312, details a number of extraction methods which could suitably be used in step (a) of the process of the present invention.

Suitably the mass of plant heated in the solvent in step (a) is at least 50 g/L, for example at least 80 g/L, preferably at least 100 g/L. Mass ratios of up to 2000 g/L, for example up to 1000 g/L or 500 g/L are suitable. Mass ratios of for example 100 g/L to 400 g/L may be used.

Step (b) comprises treating the portion of plant or an extract obtained therefrom to remove fatty acid residues from said portion of plant or extract thereof.

By fatty acid residues we mean to refer to compounds comprising fatty acids, that is long chain (for example greater than 4 carbon atoms) aliphatic moieties including an acid functionality. The fatty acid residues may be present as the free acid, salts or esters thereof, including monoesters, diesters and triesters. Phospholipids may also be present. Most commonly fatty acid residues are present as glycerol triesters.

Steps (a) and (b) may be carried out in any order. In some embodiments they may be carried out simultaneously. Preferably they are carried out sequentially. Preferably step (b) is carried out after step (a). Preferably step (b) comprises treating the extract obtained in step (a) to remove fatty acid residues.

In embodiments in which step (a) is carried out first, the extract obtained in step (a) suitably comprises saponin-containing compounds, fatty acid residues and the extraction solvent. The extraction solvent may be removed, for example under reduced pressure to provide a concentrated extract comprising saponin-containing compounds and fatty acid residues. This concentrated extract may further comprise other constituents, for example one or more of free sugars, acetylenic compounds, proteins, flavanoids, chlorophyll and lignocellulosic compounds. However these other constituents are suitably present in minor amounts, for example less than 25 wt %, preferably less than 10 wt %, more preferably less than 5 wt %. In a preferred embodiment in which the extraction solvent used comprises at least 98 wt % ethanol, and the portion of plant comprises the fruits of Hedera Helix, the concentrated extract comprises from 20 to 60 wt %, for example 35 to 45 wt % saponin-containing compounds and from 40 to 80 wt %, for example 55 to 65 wt % fatty acid residues.

When the process is carried out on the fruits of Hedera Helix, the weight ratio of saponin-containing compounds to fatty acid residues in the extract obtained in step (a) is suitably from 5:1 to 1:5, preferably from 2:1 to 1:2. This material may itself be of commercial utility as a source of saponin-containing compounds, and could be used, where appropriate in any of the applications described herein.

When the process is carried out on the leaves of Hedera Helix, the weight ratio of saponin-containing compounds to fatty acid residues in the extract obtained in step (a) is suitably from 50:1 to 1:1, preferably from 30:1 to 5:1. This material may itself be of commercial utility as a source of saponin-containing compounds, and could be used, where appropriate, in any of the applications described herein.

In some embodiments in which step (a) comprises heating the portion of plant in the extraction solvent, when the extract obtained in step (a) is allowed to cool, the fatty acid residues become less soluble and come out of solution. A separate layer may be observed forming in the extract, typically a lower layer which suitably comprises glycerol triesters of fatty acid compounds. These materials may be soluble in hot solvent, for example ethanol but become less soluble as the extract cools. Thus step (b) may comprise leaving the extract to cool. Preferably the extract is allowed to cool slowly. Once an oily layer has formed, for example at the bottom of the extract, this can be readily separated leaving a saponin-rich component in the settling vessel. Alternatively the saponin-rich component may be decanted off.

In some cases the volume of solvent in the extract obtained in step (a) may be reduced, for example by at least 25% or at least 50%, prior to carrying out step (b).

In an alternative embodiment step (b) may comprise concentrating the extract obtained in step (a) either partially or substantially to dryness and then washing the residue with a solvent having a lower polarity than the extraction solvent. Suitable solvents include hexane or other hydrocarbons, mixtures of hydrocarbons (for example those commonly known as petroleum ether), diethyl ether, ethyl acetate, and halogenated solvents (for example dichloromethane or chloroform) and acetone. Suitably the extract obtained in step (a) is first concentrated by removal of the solvent in vacuo.

In some embodiments of the first aspect of the present invention, step (b) may be carried out before step (a). In such embodiments the portion of plant is suitably treated to remove fatty acid residues and then the same portion of plant is subjected to step (a).

In embodiments in which step (b) is carried out first, a portion of plant, which is preferably in comminuted form and dried as described above, is suitably treated with a solvent in which fatty acid residues have a higher solubility than do saponin-containing compounds. Preferably fatty acid residues are substantially soluble in said solvent and saponin-containing compounds are substantially insoluble. Preferred solvents are hexane and mixtures of hydrocarbons, especially mixtures of alkanes, for example those having a boiling point of 40-80° C. Suitably in such embodiments, the portion of plant is heated in the solvent, for example at reflux, typically for a period of 1 to 24, for example 2 to 4 hours.

In some embodiments a portion of plant is heated in a series of solvents of increasing polarity incorporating as such steps (a) and (b). For example, the portion of plant may be heated first in hexane, then dichloromethane, followed by ethyl acetate and then ethanol. The ethanol fraction would be expected to be rich in saponin-containing compounds, with fatty acid residues having been extracted using the previous solvents.

In some embodiments the process of the present invention further comprises repeating steps (a) and/or step (b).

In embodiments of the process of the first aspect of the present invention in which step (b) comprises removing fatty acid residues from the extract obtained in step (a), preferably at least 50 wt % of fatty acid residues originally present in the extract are removed, preferably at least 70 wt %, more preferably at least 80 wt %, preferably at least 90 wt % and most preferably at least 95 wt %.

Suitably in step (b), along with removal of fatty acid residues, there is concurrent removal of other non-saponin species.

An advantage of the process of the present invention is that the fatty acid residues removed in step (b) are themselves of considerable commercial utility. Thus the removal of fatty acid residues in step (b) could be regarded as a separation of saponin-containing compounds and fatty acid residues.

Thus in some preferred embodiments, the process of the present invention provides a method of obtaining a saponin-rich component and a fatty acid-rich component from a plant of the Araliaceae family using a single extraction procedure.

The crude fatty acid component obtained in step (b) may include fatty acid residues of petroselinic acid, vaccenic acid and palmitoleic acid. If the portion of plant consists essentially of whole ripe fruit of Hedera Helix, it would be expected that these three acids would each be present in an amount of 20 to 40 wt % as the free acid or an ester thereof, especially a glycerol triester of one or more of these acids. This crude fatty acid component may find utility as a biofuel for example biodiesel, a dietary additive, a nutraceutical, a cosmetic base, a lubricant, or feedstock for industrial processes, for example the manufacture of surfactants. The ozonolysis products of these materials may also be of commercial utility. For example ozonolysis of petroselinic acid provides adipic acid, a precursor to nylon; and lauric acid which is used to make the surfactant sodium lauryl sulphate. Fatty acid esters obtained in step (b) could also be interesterified with other triglyceride stocks to formulate specialised mixtures having applications in food manufacture.

Some members of the Araliaceae plant family have been found to contain high concentrations of compounds of petroselinic acid. The fruits and in particular the seeds of Hedera helix have been found to include high concentrations of esters of petroselinic acid. Levels are highest when ripe or mature fruit are used.

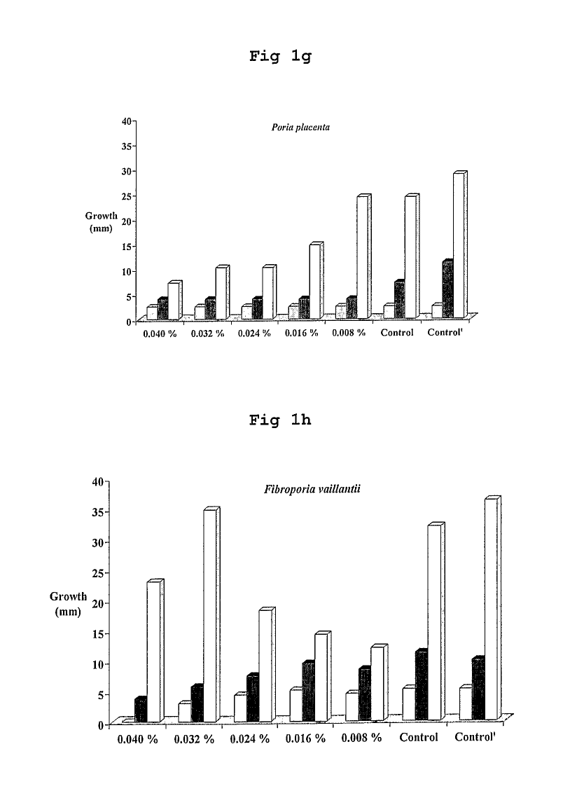

Petroselinic acid has the formula shown in FIG. 4.

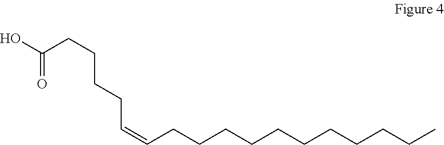

Petroselinic acid is a useful material. It is monounsatured but has similar physical characteristics to saturated fatty acids at room temperature. Petroselinic acid and derivatives thereof, especially glycerol triesters, may be used to replace saturated fats in, for example, dietary applications. It may also be used as a substitute for partially hydrogenated fats. Partially hydrogenated fats often include a double bond having a trans configuration. These are known to be damaging to human health if ingested on a regular basis.

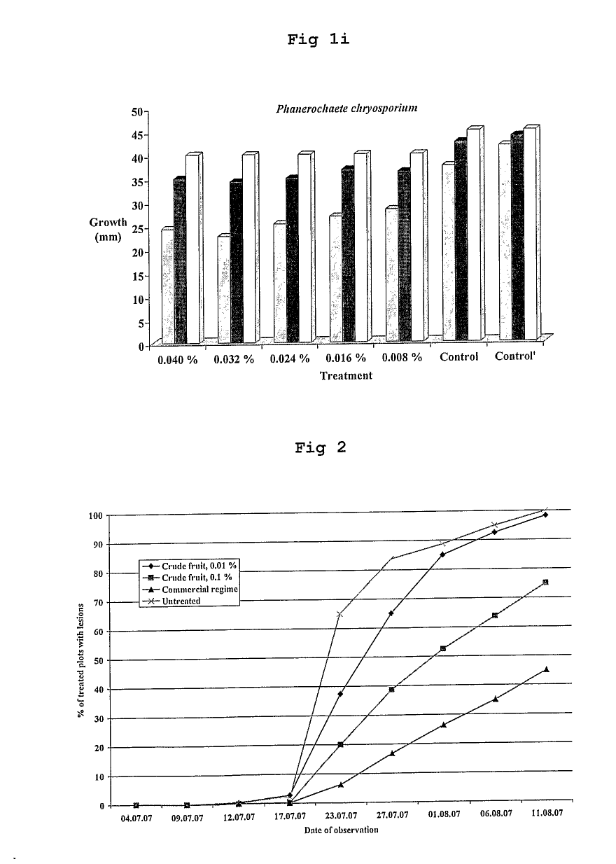

The present inventors have found that species of the Araliaceae family, in particular the fruits, and especially the seeds of Hedera helix, include high concentrations of the glycerol triester of petroselinic acid, known as tripetroselinin, that is the compound having the formula shown in FIG. 5. Indeed the present inventors have found that the seeds of Hedera helix may contain up to 80 wt % of triglycerides comprising petroselinic acid.

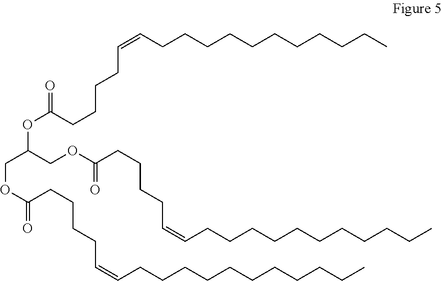

Previous methods of obtaining this compound from natural sources involved extracting the petroselinic acid as a free acid (after hydrolysis), along with other fatty acids; followed by a complex separation of petroselinic acid from the other fatty acids; and then esterifying to the glycerol triester. In another method of the prior art, tripetroselinin was recovered by molecular distillation, although the yield was poor. Petroselinic acid has been obtained from fennel seeds by acid soap crystallisation in methanol, followed by two urea segregations. The present inventors have found that using the process of the present invention, it is possible to obtain the glycerol triester of petroselinic acid in crystalline form without the need for hydrolysis and esterification, molecular distillation, interesterification, or the use of a co-crystallisation agent such as urea.

The process of the present invention may thus also be regarded as a process of obtaining a component rich in fatty acid residues. This component is obtained in step (b) of the process and is useful as a crude mixture. The crude mixture may contain a number of compounds including principally the glycerol triesters of petroselinic acid, vaccenic acid and palmitoleic acid, as well as mixed glycerol triesters of two or three of these acids. In some embodiments the crude mixture may be purified by methods known to those skilled in the art to provide the constituent fatty acids and/or esters thereof. In particular the process of the present invention is useful for providing the glycerol triester of petroselinic acid, the glycerol triester of vaccenic acid, the glycerol triester of palmitoleic acid, and mixed triesters.

The glycerol triesters thus obtained could be further reacted. For example they could be hydrolysed under acidic or basic conditions to give the free acid. This free acid could then be further reacted, for example to form a monoester. Methyl esters of fatty acids are useful as biodiesel. Monoesters could alternatively be obtained by transesterification of the glycerol triesters. Such subsequent reactions could be carried out directly on the component obtained in step (b) or on the constituent triesters after separation thereof. In some cases, subsequent reaction of the mixture may assist separation. It is possible that the component obtained in step (b) comprises mixed esters in which two or more different acids are bound to a single glycerol molecule.

In particular, when step (b) of the process of the present invention is carried out on seeds from Hedera helix or an extract thereof, the resultant fatty acid residue has been found to contain high levels of glycerol esters of petroselinic acid.

The present inventors have found that the triglycerol ester of petroselinic acid may be isolated from the fatty acid containing residue obtained in step (b) as a crystalline solid. This can be achieved by dissolving the fatty acid residue in a crystallisation solvent with or without heating, and then cooling the resultant solution to a temperature of less than 5° C., preferably less than 0° C., suitably preferably less than −5° C., for example −10° C. or −20° C. A crystalline product forms which can be collected by filtration, decanting or centrifuge and recrystallised if necessary to increase the purity thereof. The filtrate from the initial crystallisation may be rich in other fatty acid derivatives, including compounds containing residues of vaccenic and palmitoleic acids, for example the glycerol triester of vaccenic acid and the glycerol triester of palmitoleic acid. Other triglyceride components may be isolated separately from the residue obtained in step (b).

Any suitable solvent may be used as the crystallisation solvent. Suitable solvents include ketones, for example acetone; alcohols, for example ethanol; ethers, for example tetrahydrofuran or diethyl ether; esters, for example ethyl acetate; cholorinated solvents, for example dichloromethane; and alkanes, for example hexane or heptane and mixtures of alkanes. A preferred crystallisation solvent is acetone.

Preferably the glycerol triester of petroselinic acid formed by this method is at least 60% pure, preferably at least 70%, more preferably at least 90% and most preferably at least 95% pure, for example at least 99% pure.

A similar method may be used to obtain petroselinic acid as the free acid in high purity. The crude fatty acid residue obtained in step (b) may be hydrolysed under acidic or basic conditions to provide a mixture of free fatty acids. This mixture may be dissolved in a crystallisation solvent, with or without heating and cooled to a temperature of less than 5° C., for example less than 0° C. or less than −5° C. Preferred crystallisation solvents are as described above. The resultant precipitate, which may be collected by filtration, will be rich in petroselinic acid, and can be recrystallised to further increase the purity. It is thus possible to obtain petroselinic acid having a purity in excess of 70%, for example in excess of 90% or 95%.

According to a second aspect of the present invention there is provided a saponin-rich component comprising saponin-containing compounds obtainable from the Araliaceae family of plants.

Preferably the component of the second aspect is obtained from a plant of the Araliaceae family, preferably from Hedera helix. More preferably it is obtained from the fruits or the leaves of Hedera helix. Most preferably it is obtained from the fruits. It may be obtained from the whole fruit including the fleshy pulp part of the fruit and the seeds or from the fleshy pulp of the fruit alone.

Preferably the component of the second aspect is obtained by the process of the first aspect.

Preferably the saponin-rich component comprises less than 10 wt % fatty acid residues.

Preferably the component comprises less than 8 wt % fatty acid residues, more preferably less than 5 wt %, preferably less than 3 wt %, for example less than 2 wt %, preferably less than 1.5 wt %, preferably less than 1 wt % and most preferably 0.5 wt %.

Suitably the saponin-rich component of the second aspect of the present invention comprises at least 20 wt % saponin compounds, preferably at least 30 wt %, more preferably at least 40 wt %, preferably at least 50 wt %, preferably at least 60 wt %, more preferably at least 70 wt % and most preferably at least 80 wt %. By saponin compounds, we mean to refer to all compounds which include a central saponogenic core, for example the aglycone core of FIG. 1. The molecules may be further substituted with pendant side groups. The component of the second aspect preferably comprises compounds which have the structures shown in FIGS. 2 and 3 and those which include sugar residues bound to one or more of the hydroxy moieties. Compounds present include monodesmosides in which a single sugar is pendant, for example at the C3 position, and bidesmosides in which two sugar residues are found, for example, at the C3 and C28 positions. These pendant sugar residues may be monosaccharides, disaccharides or polysaccharides and may typically include one or more of arabinose, rhamnose, galactose, glucose, xylose, mannose and fructose. The relative proportion of monodesmosides and bidesmosides and the nature of the attached sugars depends on which part of the plant is used.

Preferably the component of the second aspect of the present invention comprises at least 40 wt % saponin-containing compounds having a triterpene structure, preferably at least 50 wt %, more preferably at least 60 wt %, for example at least 70 wt % or at least 80 wt %.

Preferably the component comprises less than 20 wt % of steroidal terpenes, preferably less than 10 wt %, more preferably less than 5 wt %, preferably less than 2.5 wt %, preferably less than 1 wt % and most preferably less 0.5 wt % steroidal terpenes.

Other preferred features of the second aspect of the present invention are as defined in relation to the first aspect.

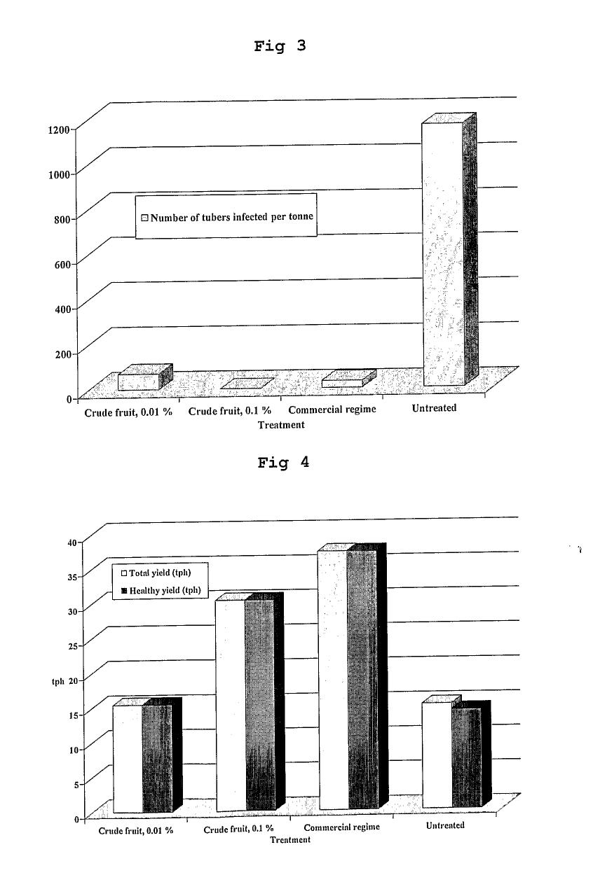

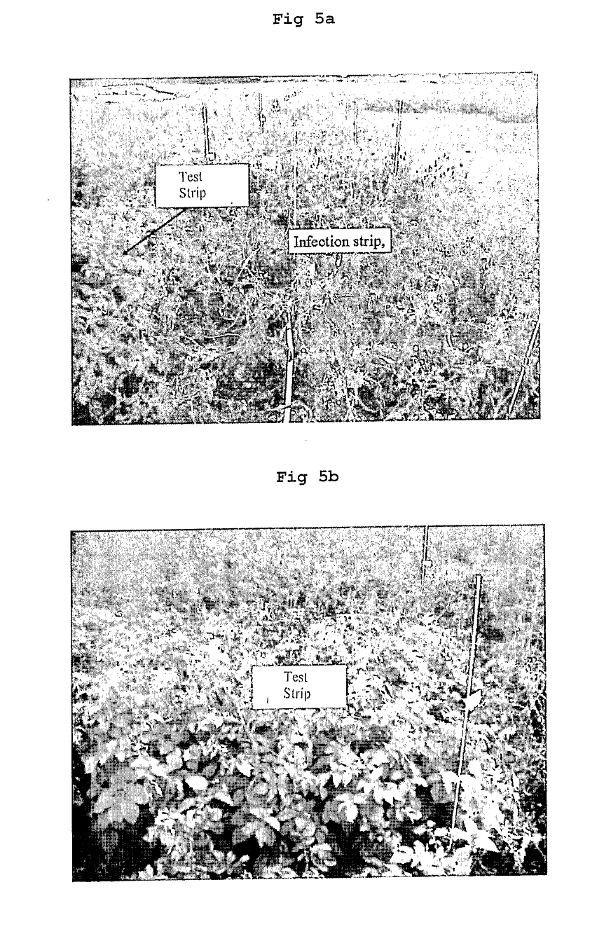

According to a third aspect of the present invention there is provided the hydrolysis product of the component of the second aspect. Suitably said hydrolysis product includes compounds having the formula showing in FIG. 6 and/or compounds having the formula shown in FIG. 7.

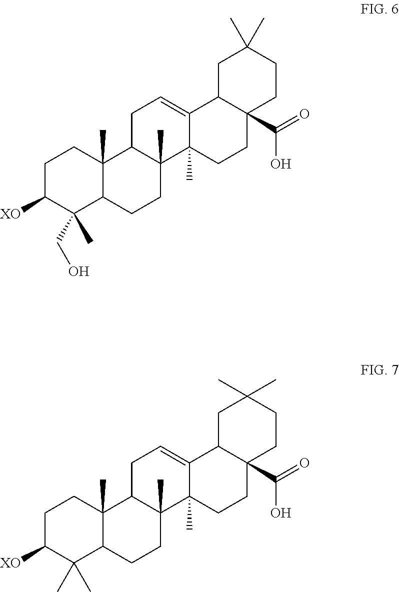

wherein X may be hydrogen or a sugar. Suitable sugars include monosaccharides, disaccharides and oligosaccharides. The sugars when present may suitably include one or more of arabinose, rhammose, galactose, glucose, xylose, mannose and fructose.

When the hydrolysis product is obtained by base hydrolysis, then in the compounds shown in FIGS. 6 and 7, X is suitably a sugar residue.

When the hydrolysis product is obtained by acid hydrolysis, then in the compounds shown in FIGS. 6 and 7, X is suitably hydrogen.

In some preferred embodiments in which the second component comprises compounds obtainable from the fruit of Hedera Helix, the hydrolysis product of the third aspect comprises at least 50 wt % of compounds having the formula of FIG. 6, preferably at least 75 wt % more preferably at least 90 wt %, for example at least 95 wt %.

The hydrolysis product of the third aspect may be formed by acid hydrolysis or by base hydrolysis. Suitably the hydrolysis product of the third aspect may be formed by suspending or dissolving the component of the second aspect is in a solvent comprising an acid or base at a concentration of 20 to 500 gdm⁻³, for example 100 to 300 gdm⁻³. Any suitable solvent can be used as will be readily known to the person skilled in the art. Examples include water and alcohols.

Suitable acids which may be used include dilute mineral acids for example hydrochloric acid, sulphuric acid, phosphoric acid and the like; and organic acids, for example formic acid, acetic acid or tosic acid and the like.

Typically the acid will be used at a concentration of 0.01 to 5 M, preferably 0.1 to 3 M.

Suitable bases which may be used include alkali metal or ammonium hydroxide (especially sodium hydroxide and potassium hydroxide) and alkali metal carbonates or ammonium carbonate.

Typically the base will be used at a concentration of 0.01 to 5 M, preferably 0.1 to 3 M.

Suitably the hydrolysis products may be obtained by stirring the suspension/solution of the component of the second aspect with the acid/base at a temperature of 30 to 100° C., for example for a period of 0.5 to 24 hours, preferably 4 to 16 hours.

Preferably the hydrolysis product of the third aspect is obtained from the component of the second aspect by hydrolysis under basic conditions.

In some embodiments a base hydrolysis step may be carried out followed by an acid hydrolysis step.

Preferably the hydrolyis product of the third aspect is obtained by the acid or base hydrolysis of the second aspect. However in some alternative embodiments, the hydrolysis product of the third aspect may be obtained directly in the method of the first aspect by including an acid or a base in the extraction solvent used in step (a).

Preferred features of the third aspect are as defined in relation to the second aspect. In particular, it is especially preferred that the hydrolysis product is obtained from the fruits of Hedera Helix.

According to a fourth aspect of the present invention there is provided a composition comprising the component of the second aspect, the hydrolysis product of the third aspect, or a mixture thereof.

The composition of the fourth aspect is preferably an aqueous composition.

The composition of the fourth aspect may comprise the component of the second aspect, the hydrolysis product of the third aspect or a mixture thereof.

In some preferred embodiments, the component of the second aspect is not present in the composition.

In some preferred embodiments, the hydrolysis product of the third aspect is not present in the composition.

In some embodiments the composition of the fourth aspect may be prepared by dissolving the component of the second aspect and/or the hydrolysis product of the third aspect in a small volume of water-miscible solvent (for example an alcohol) in which it is readily soluble and then diluting with water to form a substantially aqueous composition.

The fourth aspect of the present invention may suitably provide a composition comprising from 0.00001 to 5 wt % of saponin-containing compounds obtainable from plants of the Araliaceae family, especially Hedera helix. In some preferred embodiments the composition comprises compounds obtainable from the fruit of Hedera Helix. In some preferred embodiments the composition comprises compounds obtainable from the leaves of Hedera Helix.

The invention may also provide a concentrated formulation which upon dilution forms a composition of the fourth aspect. This may be provided in the form of a solid or liquid, for example a powder, a suspension, a solution or an emulsion.

The composition of the fourth aspect may suitably be varied according to the intended use thereof. It may further comprise one or more components selected from a water miscible solvent (for example ethanol, propanol or polyethylene glycol), an antioxidant (for example tocopherol or BHT), a chelating agent (for example EDTA), a dispersant, a surfactant and an emulsifier.

In some embodiments the composition may further comprise an adhesion-promoting agent. This may be included, for example in an aqueous composition which is applied to a crop.

Suitable adhesion-promoting agents are materials which enable the composition to stick to a substrate more readily. The substrate may for example be a plant and an adhesion-promoting agent will help prevent the composition from running off the plant and/or being washed away in rainfall. Suitable compounds for use as adhesion-promoting agents include natural rubber latex or synthetic latex.

The adhesion-promoting agent is preferably present in the composition in an amount of from 0.001 to 5 wt %, for example 0.005 to 2 wt %, preferably 0.01 to 0.2 wt %.

Other preferred features of the fourth aspect are preferably as defined in relation to the first and/or second and/or third aspects.

The present inventors have found that the saponin-containing components of the present invention may have molluscicidal or molluscal antifeedent or molluscal repellent properties. They are particularly effective against terrestrial molluscs.

According to a fifth aspect of the present invention there is provided the use of the composition of the fourth aspect as a molluscicide and/or a mollusc repellent and/or a mollusc antifeedant.

By molluscicide, we mean to refer to a material which when applied to an area (e.g. a plant) will cause molluscal species that ingest the material to be killed. Suitably the molluscicide kills more than 30%, preferably 50% of molluscs which ingest it within 1 hour.

By mollusc antifeedant we mean to refer to a material which when applied to an area (for example a plant) prevents molluscs from feeding in that area. A suitable mollusc antifeedant is a material which when applied to a plant reduces consumption of that plant by a mollusc over a period of 12 hours by at least 10%, preferably at least 20%, more preferably at least 50%, most preferably at least 90%.

By mollusc repellent we mean to refer to a material which repels molluscs. Thus if an area is treated with a material which behaves as a mollusc repellent, molluscs would preferentially not enter this area. Suitably a 50% reduction in mollusc entry would be seen.

When used as a molluscicide and/or mollusc antifeedant and/or mollusc repellent, the composition of the fourth aspect may be provided in any suitable form. It may, for example, be provided as an aqueous composition. Alternatively it may be provided in the form of pellets, as a powder or bound with a polymer.

In aqueous compositions suitable for spraying onto a crop as a molluscicide and/or mollusc antifeedant and/or mollusc repellent, the component of the second aspect or the hydrolysis product of the third aspect is present preferably in an amount of from 0.001 to 5 wt %, preferably 0.01 to 2.5 wt %, more preferably 0.05 to 2 wt %, for example 0.5 to 1.5 wt %.

When the molluscicide and/or mollusc antifeedant and/or mollusc repellent composition is in the form of a powder, it may suitably comprise an inert filler, for example vermiculite and/or a dessicant, for example silica gel. Such powders are typically dusted on or around plants to be protected.

When the composition is provided in the form of pellets, it preferably comprises a mollusc attracting material. Suitable mollusc-attracting materials include bran, yeast, wheat and sugars (for example sucrose and glucose).

The present invention provides molluscicidal and/or molluscal antifeedant and/or molluscal repellent pellets comprising the component of the second aspect or the hydrolysis product of the third aspect. Preferably the pellets comprise from 0.1 to 25 wt %, for example 1 to 15 wt %, preferably 4 to 10 wt % of the component of the second aspect or the hydrolysis product of the third aspect. The pellets may comprise the concentrated extract obtained in step (a) of the process of the first aspect, or may include whole fruit and/or leaves of Hedera Helix.

The pellets may suitably further comprise one or more of fillers, bran, wheat, yeast, sugars, emulsifiers and antifungal agents. A typical slug pellet of the present invention comprises 90 to 95 wt % bran, wheat or a mixture thereof, 0.1 to 2 wt % yeast, 0.5 to 2 wt % sugars, 0.0005 to 0.005 wt % antifungal agent, 0.01 to 0.1 wt % polysorbate emulsifier (for example as sold under the trade mark Tween 80) and from 1 to 8 wt % of the component of the second aspect, or the hydrolysis product of the third aspect.

Slug pellets of the present invention may further comprise an additional known molluscicide, for example metaldehyde, methiocarb or thiodicarb.

In another embodiment the present invention may provide a polymeric material having bound thereto or within saponin-containing compounds obtainable from the Araliaceae family of plants. The saponin-containing compounds may, for example be covalently bound to a polymeric residue. In such embodiments the polymer may be formed into a plastic plant mat or band which could be attached to a plant that is desired to be protected against molluscs.

In an alternative embodiment the polymer may comprise a latex-like material having dispersed therein saponin-containing compounds. This may be applied to ligno-cellulosic materials, for example wood fibre, or other material, for example agricultural wastes formed into fibre as mats to bind the ligno-cellulosic materials. These may be laid on or around the plants. The plants may also be planted directly through the mats.

The component of the second aspect or the hydrolysis product of the third aspect could be incorporated in a polymeric seed coating which would allow the seed to germinate but would provide molluscicidal and/or molluscal antifeedant and/or molluscal repellent properties. Techniques for coating seeds in such a manner include prilling, soaking and spraying. These and other methods are understood by those skilled in the art.

The present inventors have discovered that the component of the second aspect and the hydrolysis product of the third aspect of the present invention show significant advantages in combating late potato blight.

According to a sixth aspect of the present invention there is provided the use of a composition of the fourth aspect in combating late potato blight.

By combating late potato blight we mean to include preventing the occurrence of, inhibiting the growth of and controlling late potato blight. Late potato blight is also known as Phytophthora infestans.

Thus the present invention further provides a method of combating late potato blight, the method comprising applying to a crop a composition of the fourth aspect.

The method may be used on any crop infested with phytophthora infestans. Suitably the crop may be selected from a potato crop and a tomato crop.

Suitably the method comprises applying a composition comprising from 0.0001 to 5 wt %, for example 0.0005 to 1 wt %, preferably 0.001 to 0.05 wt %, more preferably 0.001 to 0.25 wt % of the saponin-containing component of the second aspect or the hydrolysis product of the third aspect of the present invention to the crop.

Suitably in the method of combating potato blight of the present invention the composition of the fourth aspect is applied to the crop once every 3 to 12 days, for example once every 5 to 9 days, for example every 6 to 8 days, for example every 7 days.

Typically a crop will be treated for a period of at least 4 weeks, preferably a period of at least 6 weeks, for example for a period 8 to 16 or 10 to 12 weeks.

The method of combating potato blight of the present invention may include treating the crop on some occasions with a composition of the fourth aspect and on other occasions with a composition comprising a different agent able to combat potato blight. In some embodiments a composition of the fourth aspect and a composition comprising another agent able to combat potato blight may be coapplied. Suitably in the method of the present invention, between 1 in 2 and 1 in 6 treatments of a crop may include applying a composition of the fourth aspect.

In the method of combating potato blight of the present invention, the composition may be applied to the crop by any suitable means known to those skilled in the art. One suitable method is spraying the crop. Typically each hectare of crop will be sprayed with between 100 and 1000 g, preferably between 200 and 500 g, for example about 300 g of the component of the second aspect or the hydrolysis product of the third aspect. This would be applied in dilute form as a composition of the fourth aspect. Typically a dried powder composition is diluted with water and then applied to the crop, for example at a concentration of 0.1 to 10 g/L.

The crude extract obtained in step (a) of the process of the first aspect could be incorporated into a composition for combating blight.

The present inventors have also found that saponin-containing compounds obtainable from Hedera helix show fungicidal and/or fungistatic activity.

According to a seventh aspect of the present invention there is provided the use of a component of second aspect or a hydrolysis product of the third aspect or compositions comprising the same as a fungicide and/or a fungistatic agent.

The component of the second aspect and the hydrolysis product of the third aspect have been found to be effective against the fungal species Serpula lachrymans, Phlebia gigantea, Trametes versicolor, Heterobasidion annosum, Trichoderma viride, Coniophera puteana, Poria placenta, Fibroporis vaillantii, Pleurotus Ostreatus, Gloeophyllum Trabeum and Phanerochaete chryosporium. It may also be effective against other organisms, for example Alternaria, Aspergillus, Cladosporium, Botrytis, Anthracnose, Drechslera, Fusarium, Plasmopara, Pseudoperonospora, Pythium, Phytophthora, Rhizoctonia, Sclerotinia, Candida and Uredinales.

The component of the second aspect and the hydrolysis product of the third aspect have been found to be particularly effective against the Candida albicans, Phytophthora, Aspergillus fumigatus, Pleurotus Ostreatus and Gloeophyllum Trabeum.

In particular the present inventors have found that the hydrolysis product of the third aspect obtained by base hydrolysis is especially effective against the Candida fungal species.

The present invention is particularly effective at combating fungi that commonly grow on cut timber. By combating fungus we mean to include preventing the growth of fungus, inhibiting the growth of fungus already present and killing fungus already present.

The present invention therefore provides a method of combating fungal growth on timber, the method comprising applying to the timber a composition of the fourth aspect.

Preferably the composition for applying to cut timber comprises from 0.0001 to 5 wt %, more preferably 0.0005 to 2.5 wt, preferably 0.001 to 1 wt %, most preferably 0.005 to 0.5 wt % of a component of the second aspect or the hydrolysis product of the third aspect. The method is effective against timber decay fungi.

Preferably the method comprises applying a composition to the timber by spraying, coating, painting, dipping, impregnation under pressure or other methods known to those skilled in the art. A single application may be effective or in some cases a plurality of applications may be necessary.

The present invention may also provide the use of component of the second aspect or the hydrolysis product of the third aspect as an adjuvant in fungal control.

By an adjuvant in fungal control, we mean to refer to a secondary component that is added to an existing fungicide composition to improve the efficacy thereof. The component of the second aspect or the hydrolysis product of the third aspect of the present invention may be added to a fungicidal composition comprising one or more approved plant protection or biocidel products, including Myclobutanil, Cyclohexadone, Mancozeb, Oxycarboxin, Propamocarb hydrochloride, Chlorothalonil and Etridiazole. Such a composition may typically be applied to a substrate by dipping, spraying, impregnating, covering, painting or other suitable method.

The present inventors have also found that the component of the second aspect or the hydrolysis product of the third aspect of the present invention has insecticidal and insect antifeedant and repellent properties.

Thus the present invention further provides the use of a component of the second aspect, the hydrolysis product of the third aspect or compositions comprising the same as an insecticide and/or insect antifeedant and/or insect repellent properties.

Compositions of the fourth aspect have been found to be particularly effective at combating wood boring insects, for example beetles and termites.

The invention provides the use of a component of the second aspect, the hydrolysis product of the third aspect or compositions comprising the same for combating click beetles or wireworms.

The invention provides the use of a component of the second aspect, the hydrolysis product of the third aspect or compositions comprising the same for combating nematodes.

The invention provides the use of a component of the second aspect, the hydrolysis product of the third aspect or compositions comprising the same as an anthelmintic agent for human and/or veterinary use.

The invention provides the use of a component of the second aspect, the hydrolysis product of the third aspect or compositions comprising the same as an antibacterial agent, for example for use in combating a bacterial species selected from Pseudomonas, Ralstonia, Streptomyces, Xanthomonas and Xylophilus.

The saponin-containing compounds obtainable from Araliaceae plants may be modified to provide novel chemical compounds which show improved biological activity.

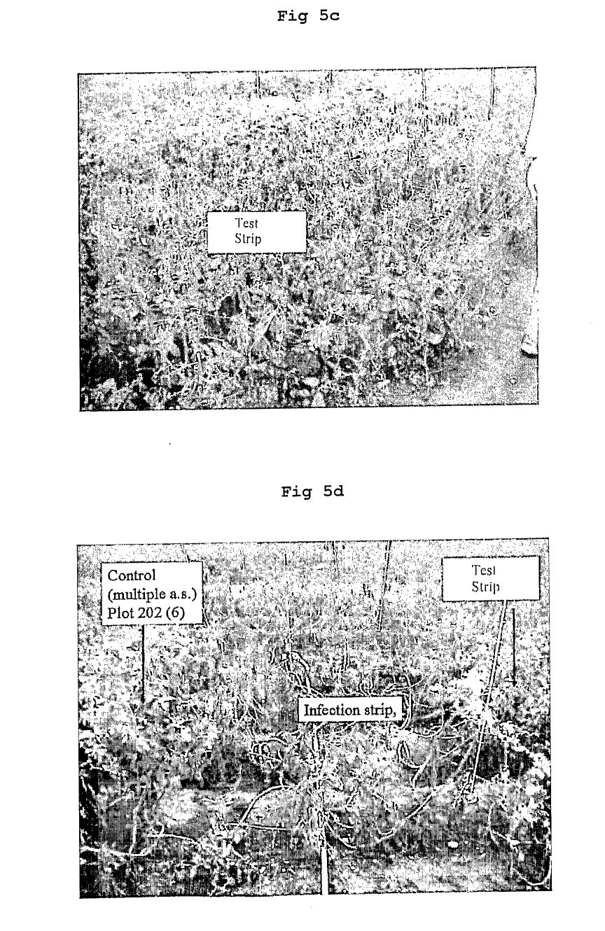

One class of compound which may be obtained in this manner is shown in FIG. 8, wherein X, Y and Z are independently selected from hydrogen, a metal ion, an optionally substituted hydrocarbyl group, a protecting group, a fatty acid residue or a sugar residue wherein at least one of X, Y and Z is not hydrogen.

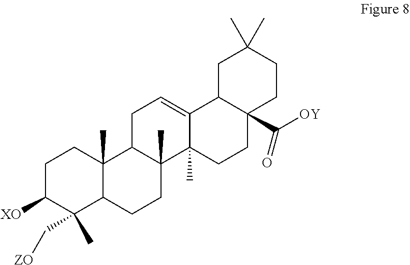

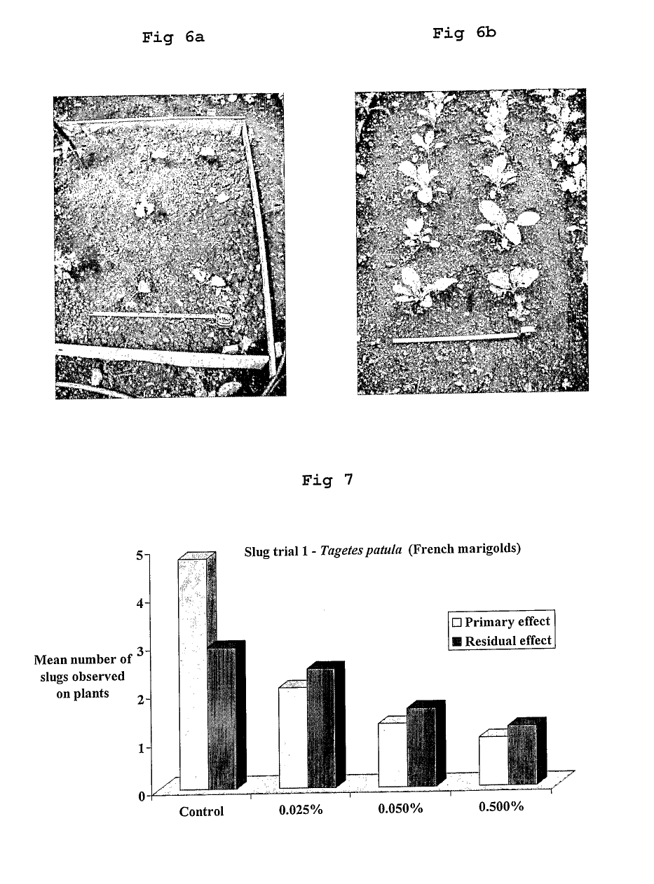

Another class of compounds which can be prepared according to the present invention is shown in FIG. 9 wherein P and Q are independently selected from hydrogen, a metal ion, an optionally substituted hydrocarbyl group, a protecting group, a fatty acid residue or a sugar residue wherein at least one of P and Q is not hydrogen.

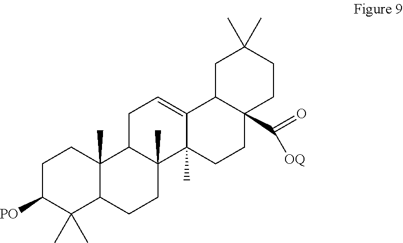

The compounds of FIGS. 8 and 9 may be further modified by reaction of the double bond and products resulting from such a reaction are also within the scope of the present invention. For example the double bond could be hydrogenated. Water or another reagent could be added across it. For example, each end of the double band may be independently substituted with, for example, H, OH, Br or Cl. The additional functionality thus introduced by could be further manipulated by subsequent reaction.

When one or more of X, Y, Z, P or Q is a metal ion, each may be independently selected from alkali metals, alkaline earth metals, transition metals, lanthanides and metals of the p-block. Preferably in such embodiments the or each of X, Y, Z, P or Q is a transition metal. Most preferably the or each of X, Y, Z, P or Q is selected from silver and copper.

By optionally substituted hydrocarbyl group, we mean to refer to an alkyl, alkenyl, alkynyl, aryl or aralkyl group which may or may not be substituted with one or more substituents, for example those selected from fluoro, chloro, bromo, hydroxy, alkyoxy, acetoxy, thiol, thioether, sulfone, sulfoxide, nitro and amino. Such groups preferably contain up to 30 carbon atoms, for example up to 20 carbon atoms, preferably up to 10 carbon atoms.

Each of X, Y, Z, P and Q may be independently selected from protecting groups commonly used to protect alcohols and/or carboxylic acids.

Suitable protecting groups include acetal, allyl, silyl ether, tetrahydropyran, acetyl, methoxymethyl ether, paramethoxylbenzyl ether, pivaloyl, methyl ethers, methyl esters, benzyl esters and tertiary butyl esters.

When any one or more of X, Y, Z, P or Q is a sugar residue, each may independently be selected from a monosaccharide, a disaccharide, a trisaccharide or polysaccharide. Alternatively X, Y, Z, P and Q may be selected from other carbohydrate residues, for example anhydrosugars.

When any one or more of X, Y, Z, P or Q is a monosaccharide, each may be independently selected from glucose, fructose, galactose, xylose, ribose, erythrose, threose, erythruylose, arabinose, lyxose, ribulose, xylulose, allose, altrose, glucose, gulose, idose, mannose, talose, fructose, psicose, sorbose, rhamnose and tagatose.

When any of X, Y, Z, P or Q is a disaccharide each may independently be selected from sucrose, lactose, maltose, trehalose and cellobiose.

Any of X, Y, Z, P or Q may be a fatty acid residue. By this we mean to include embodiments in which Z and Q are derived from the corresponding fatty alcohols and X, Y, and P are residues of the acid. Suitable fatty acids are those having from 6 to 36 carbon atoms, for example 10 to 30 carbon atoms, preferably 12 to 24 carbon atoms, more preferably 16 to 20 carbon atoms. Fatty acid residues may be branched or unbranched, and may be saturated, monosaturated or polyunsatured. They may include from 0 to 5 double bonds. The fatty acid residue may suitably be selected from butyric, caproic, caprylic, capric, lauric, myristic, palmitic, stearic, arachidic, behenic, petrolselinic, myristoleic, palmitoleic, oleic, linoleic, vaccenic, alpha-linolenic, arachidonic, eicosapentaenoic, erucic, docohexaenoic, and trans isomers of unsaturated fatty acids.

In especially preferred embodiments in which any of X, Y, Z, P or Q is a fatty acid residue, each may independently be the fatty acid residue of petroselinic acid, vaccenic acid or palmitoleic acid.

The present invention also includes methods of preparing the compounds having the structures shown in FIGS. 8 and 9.

The compounds of FIGS. 8 and 9 may be prepared by manipulation of the naturally occurring saponin-containing compounds extracted from Araliaceae plants. These could for example be obtained by the process of the first aspect. Naturally occurring saponin-containing compounds obtained from the plants typically include a sugar residue at X and/or Z or P and/or Q. This sugar residue may be removed by mineral acid or enzymatic hydrolysis (for example using the method disclosed by Hostettman and Marston in Saponins, Phillipson, J. (ed)., Chemistry and Pharmacology of Natural Products, Cambridge University Press, 1995) to provide the alglycone unit shown in FIG. 2 or 3. Enzymatic hydrolysis may be used to selectively remove alcohol or acid-bound sugar residues. Base hydrolysis could remove acid-bound sugars. Such reactions are described in the literature, see for example Hostettmann, K. Helvetica Chimica Acta, 1980, 63:3, 606-609; and Bedir, E.; Kirmizipekmez, H.; Sticher, O.; [symbol] ali [symbol] , I. Phytochemistry, 2000, 53:8, 905-909.

This unit could be manipulated by reaction at the positions shown as substituted by X, Y, Z, P, or Q or by reaction of the double bond. Reactions to add sugar and/or fatty acid compounds at these positions or to manipulate the double bond, are known to those skilled in the art.

Thus the compounds of FIGS. 8 and 9 may be prepared from or via the hydrolysis product of the third aspect.

The present invention further provides compositions comprising novel compounds having the structures shown in FIG. 8 or FIG. 9. The compositions may comprise at least 0.0001 wt % of the compounds of FIG. 8 or FIG. 9, for example at least 0.0005 wt %, at least 0.001 wt % or at least 0.005 wt %. The compositions may comprise up to 25 wt % of the compounds of FIG. 8 or FIG. 9, for example up to 10 wt %, up to 5 wt %, up to 2.5 wt % or up to 1 wt %.

Such compositions may further comprise one or more of a mollusc attracting compound, an adhesion-promoting agent, a water miscible solvent (for example ethanol, propanol or polyethylene glycol), an antioxidant (for example tocopherol or BHT), a chelating agent (for example EDTA), a dispersant, a surfactant and an emulsifier.

The present invention provides compounds having the structure of FIG. 8 or FIG. 9 for use in therapy. A further aspect provides the use of the compounds for the therapeutic treatment of humans or animals or the treatment of plants against plant pathogens. Such treatments may be preventative or curative.

The present invention further provides the use of compounds having the structure of FIG. 8 or FIG. 9 or compositions comprising the same as a molluscicide and/or a mollusc antifeedant and/or a mollusc repellent. In the use of the fifth aspect and associated methods and compositions, the component of the second aspect may be replaced with compounds having the structure shown in FIG. 8 or FIG. 9.

The invention also provides the use of compounds having the structure of FIG. 8 or FIG. 9 or compositions comprising the same in a method of combating potato blight. In the use of the sixth aspect and associated methods and compositions, the component of the second aspect may be replaced with compounds having the structure shown in FIG. 8 or FIG. 9.

The present invention provides the use of compounds having the structure of FIG. 8 or FIG. 9 or compositions comprising the same as a fungicide, a fungistastic agent or an adjuvant in fungal control. In the use of the seventh aspect and associated methods and compositions, the component of the second aspect may be replaced with compounds having the structure shown in FIG. 8 or FIG. 9.

In some embodiments the compounds of FIGS. 8 and 9 may be regarded as modified saponin compounds which saponin compounds are obtained by extraction from plants of the Araliaceae family, suitably by the method of the first aspect. The present inventors have found that such modified compounds, along with the acid hydrolysis products and base hydrolysis products of the third aspect have particularly beneficial properties.

Any feature of any aspect of the present invention may be combined with any feature of any other aspect unless it would be inconsistent to make such a combination.

The present invention will now be further described by way of the following non limiting examples.

### EXAMPLE 1

### Extraction of Dried, Prepared H. helix Fruits

Ripe fruits (15.00 kg, 69.34% moisture content) were collected from various locations on Anglesey in April 2005 and dried in a 50° C. oven for two days, reducing the moisture content (m.c.) to 4.43%. They were then minced to a meal (4.59 kg) in a food processor and a portion of the meal (94.16 g) taken for immediate extraction. The remainder was placed in an airtight container and frozen for future use.

On defrosting, two samples of prepared meal (30.00 g and 60.00 g) were extracted separately and exhaustively in a Soxhlet apparatus for 48 hours using EtOH (99%, 250 ml) at reflux. Following removal of the solvent in vacuo, the pasty solid recovered was washed in petrol (50 ml×3) and filtered through a Buchner funnel under reduced pressure. The solid recovered was dried in an oven at 50° C. overnight then recovery of crude saponin component recorded. The solvent wash was removed in vacuo and the recovery of crude fatty acid residue recorded. Table 1 shows the amount of each component recovered.

TABLE 1

| | | Recovery | | Recovery |
|---|---|---|---|---|
| | Crude | saponin (%) at | Crude | fatty acid (%) at |
| Sample | Saponin (g) | o.d.w. | fatty acid (g) | o.d.w. |
| 30 g | 7.60 | 26.51 | 7.69 | 26.82 |
| 60 g | 16.93 | 29.53 | 17.62 | 30.72 |

The presence of saponins was confirmed by ¹H-NMR spectroscopy showing typical signals of this class of compound against standards of hederagenin, α-hederin and hederacoside C.

Transesterification of the fatty acid component to the respective fatty acid butyl esters and subsequent GCMS analysis against fatty acid methyl ester standards and comparison with literature data for butyl esters showed principally petroselinic acid (30 g=28.73%, 60 g=29.37%), palmitoleic acid (30 g=20.43%, 60 g=20.16%) and vaccenic acid (30 g=16.99%, 60 g=16.84%).

By contrast, under the same conditions for recoveries of crude saponins and fatty acid components from fruits respectively, a methanol extract afforded 29.89% and 11.27% but from 2-propanol 28.73% and 33.98% were recovered.

### EXAMPLE 2

On a larger scale, fruits (26.45 kg, 67.8% m.c.), were prepared as described in relation to example 1 above to obtain dried fruits that were frozen until used. The meal was thawed and a portion (6.58 g) taken for m.c. determination, establishing 7.44%. With stirring (Heidolph RZR2102 overhead stirrer at 91 r.p.m.) the remaining dried material (7.87 kg) was charged to a 50 L jacketed vessel (Diehm, Wertheim), holding 35 L of EtOH. The vessel was heated using silicone oil and a heater unit (Huber Wright 141) to 50° C. and left, with stirring for six hours. It was then switched off and left to cool overnight. The vessel was then drained down and the solvent removed on a rotary evaporator stepwise to obtain a thick paste. 26.97 L of EtOH were recovered. The paste was washed exhaustively using 40-60 petrol. The solution was filtered through a Buchner funnel under reduced pressure and the solvent removed on a rotary evaporator to obtain a green oil (537.92 g, 6.84% o.d.w.). The remaining paste was removed from the flask to an evaporating basin then placed in a 50° C. oven for three days. After grinding in a pestle and mortar, a fine, amorphous, purple powder (1,176.20 g, 14.94%) was recovered. A ¹H-NMR spectrum of this confirmed the product was principally saponins.

The recovered solvent was returned to the vessel and the extraction repeated at 50° C. without stirring as the solids had compacted. The solvent sat above the filter cake and percolated through slowly. It was drained down over two days and treated as above to recover EtOH (21.06 L), a green oil (322.83 g, 4.10% o.d.w.) and a brown solid (384.05 g, 4.88% o.d.w.). ¹H-NMR of the solid confirmed the saponins were present as the principal component. The recovery of fatty acid residues can be improved by draining of the vessel at higher temperatures. Crude saponin recovery at nearly 20% is less than that obtained at Soxhlet conditions though had the solvent been drawn down hot, recovery of both components would have been improved.

### EXAMPLE 3

### Fungistatic Action on Timber Decay Fungi

Five concentrations containing respectively 0.10 g, 0.08 g, 0.06 g, 0.04 g, and 0.02 g of the crude saponin extract obtained in example 1 were made with identical preparation of two controls. The extract was suspended in tap water (10 ml) using a sonic bath to disperse the solids. This was made up with water (250 ml) containing 2% malt agar then autoclaved. The growth of the following fungi was observed: Serpula lachrymans, Phlebia gigantea, Trametes versicolor, Heterobasidion annosum, Trichoderma viride, Coniophera puteana, Poria placenta, Fibroporis vaillantii and Phanerochaete chryosporium. Plugs (approx 5 mm²) taken from fungal colonies were placed on the agar and the plates left to develop. Observations were made at 3, 6 and 27 days, recording growth (in mm) N, S, E & W on an 80 mm Petri dish, with the exception of Serpula lachrymans where observations were made on days 6 and 27. The results are shown in FIGS. 1a-1i.

### EXAMPLE 4

Due to the low solubility of the crude extract in water, the experiment was repeated, replacing the step in example 3 of taking up the product in water with dissolving the solids in EtOH (10 ml, as IMS 99). In this case the amounts of extract used were 0.32 g, 0.16 g, 0.08 g, 0.04 g and 0.02 g. Daily observations were made for 28 days and recorded as previously. The results are shown Table 2.

TABLE 2
Trametes versicolor

| | | | | | | |
|---|---|---|---|---|---|---|
| | 2 | 4 | 6 | 8 | 10 | 13 |
| | days | days | days | days | days | days |
| Control | 0.25 | 6.00 | 11.00 | 19.00 | 28.00 | 40.00 |
| 0.008% | 0.25 | 3.75 | 10.25 | 16.50 | 22.50 | 33.25 |
| 0.016% | 0.00 | 4.00 | 9.75 | 16.00 | 22.50 | 32.00 |
| 0.032% | 0.00 | 2.00 | 7.00 | 14.00 | 20.25 | 29.75 |
| 0.064% | 0.00 | 3.25 | 9.25 | 15.00 | 22.75 | 34.50 |
| 0.128% | 0.00 | 1.00 | 11.25 | 17.00 | 26.00 | 35.50 |

Coniophora puteana

| | | | | | | | | |
|---|---|---|---|---|---|---|---|---|
| | 2 | 4 | 6 | 8 | 10 | 12 | 14 | 16 |
| | days | days | days | days | days | days | days | days |
| Control | 2.50 | 9.25 | 18.00 | 27.00 | 40.00 | 49.38 | 58.76 | 68.14 |
| 0.008% | 1.25 | 4.50 | 11.00 | 15.67 | 22.50 | 28.25 | 32.00 | 40.00 |
| 0.016% | 0.25 | 2.25 | 7.50 | 13.50 | 19.50 | 25.25 | 30.00 | 37.25 |
| 0.032% | 0.25 | 2.00 | 5.00 | 11.25 | 17.50 | 24.50 | 29.50 | 38.50 |
| 0.064% | 0.25 | 2.25 | 5.25 | 11.00 | 17.50 | 24.00 | 31.25 | 36.00 |
| 0.128% | 0.00 | 0.75 | 5.00 | 11.25 | 17.50 | 23.50 | 29.50 | 34.00 |

Trichoderma viride

| | | | | | |
|---|---|---|---|---|---|
| | 2 | 4 | 6 | 8 | 10 |
| | days | days | days | days | days |
| Control | 9.00 | 23.00 | 37.25 | 54.12 | 68.24 |
| 0.008% | 5.25 | 11.33 | 15.00 | 24.33 | 31 |
| 0.016% | 3.50 | 11.00 | 17.75 | 24.25 | 30 |
| 0.032% | 3.00 | 10.25 | 16.75 | 22.75 | 28.25 |
| 0.064% | 2.75 | 10.00 | 15.33 | 22.00 | 26.5 |
| 0.128% | 4.00 | 15.00 | 15.00 | 22.50 | 26 |

Poria placenta

| | | | | | | | | | | |
|---|---|---|---|---|---|---|---|---|---|---|
| | 2 | 5 | 8 | 11 | 14 | 17 | 20 | 23 | 26 | 28 |
| | days | days | days | days | days | days | days | days | days | days |
| Control | 0.00 | 0.00 | 5 | 7 | 10.00 | 14.00 | 20.00 | 26.25 | 28.50 | 30.50 |
| 0.008% | 0 | 0.00 | 1 | 2 | 3 | 4.00 | 5.00 | 6.00 | 7.00 | 8.00 |
| 0.016% | 0 | 0.00 | 0.5 | 2 | 2.25 | 3.50 | 5.00 | 7.25 | 7.75 | 10.50 |
| 0.032% | 0 | 0.00 | 0 | 1 | 1 | 2.25 | 3.25 | 5.25 | 6.50 | 8.00 |
| 0.064% | 0 | 0.00 | 0 | 1 | 1.75 | 3.50 | 4.75 | 9.00 | 9.25 | 11.25 |
| 0.128% | 0 | 0.00 | 0 | 1.25 | 1.5 | 2.25 | 3.75 | 6.50 | 7.25 | 9.25 |

Phanerochaete chryosporium

| | | | | | | | | |
|---|---|---|---|---|---|---|---|---|
| | 2 | 5 | 8 | 11 | 14 | 17 | 20 | 22 |
| | days | days | days | days | days | days | days | days |
| Control | 2.38 | 9.00 | 21.00 | 26.00 | 40.00 | 49.42 | 58.84 | 65.12 |
| 0.008% | 1.50 | 6.00 | 10.00 | 15.00 | 20.00 | 25.00 | 27.75 | 33.00 |
| 0.016% | 2.75 | 6.00 | 11.75 | 17.50 | 24.00 | 27.50 | 33.00 | 33.25 |
| 0.032% | 1.00 | 5.00 | 9.50 | 14.00 | 22.00 | 26.75 | 31.50 | 31.75 |
| 0.064% | 0.13 | 4.50 | 8.75 | 13.75 | 20.50 | 25.75 | 30.25 | 35.00 |
| 0.128% | 0.00 | 3.25 | 7.00 | 12.25 | 17.00 | 20.00 | 22.00 | 30.50 |

Phlebia gigantea

| | | | | | | | | | | |
|---|---|---|---|---|---|---|---|---|---|---|
| | 2 | 5 | 8 | 11 | 14 | 17 | 20 | 23 | 26 | 28 |
| | days | days | days | days | days | days | days | days | days | days |
| Control | 1.00 | 5.00 | 13 | 18.75 | 30.00 | 40.84 | 48.10 | 55.36 | 62.62 | 67.46 |
| 0.008% | 0.00 | 3.50 | 6 | 9.00 | 12.50 | 15.25 | 17.00 | 20.25 | 22.00 | 23.25 |
| 0.016% | 0.00 | 3.00 | 6 | 8.25 | 11.00 | 12.25 | 15.00 | 17.25 | 19.00 | 20.75 |
| 0.032% | 0.00 | 2.00 | 5 | 7.00 | 9.75 | 13.00 | 15.75 | 21.00 | 24.00 | 27.00 |
| 0.064% | 0.00 | 1.00 | 3.75 | 7.00 | 8.75 | 13.00 | 17.75 | 21.75 | 24.00 | 26.75 |
| 0.128% | 0.00 | 1.25 | 3.5 | 7.00 | 9.25 | 12.25 | 16.25 | 18.50 | 21.75 | 25.75 |

Fibroporia vaillanti

| | | | | | | | | | | |
|---|---|---|---|---|---|---|---|---|---|---|
| | 2 | 5 | 8 | 11 | 14 | 17 | 20 | 23 | 26 | 28 |
| | days | days | days | days | days | days | days | days | days | days |
| Control | 0.00 | 0.00 | 0 | 3 | 4.00 | 11.00 | 18.00 | 24.00 | 28.00 | 33.75 |
| 0.008% | 0.00 | 0.00 | 0 | 0 | 0 | 0.00 | 0.00 | 0.00 | 0.00 | 0.00 |
| 0.016% | 0.00 | 0.00 | 0 | 0 | 0 | 0.00 | 0.00 | 0.00 | 0.00 | 0.00 |
| 0.032% | 0.00 | 0.00 | 0 | 0 | 0 | 0.00 | 0.00 | 0.00 | 0.00 | 0.00 |
| 0.064% | 0.00 | 0.00 | 0 | 0 | 0 | 0.00 | 0.00 | 0.00 | 0.00 | 0.00 |
| 0.128% | 0.00 | 0.00 | 0 | 0 | 0 | 0.00 | 0.00 | 0.00 | 0.00 | 0.00 |

Heterobasidion annosum

| | | | | | | | | | | |
|---|---|---|---|---|---|---|---|---|---|---|
| | 2 | 5 | 8 | 11 | 14 | 17 | 20 | 23 | 26 | 28 |
| | days | days | days | days | days | days | days | days | days | days |
| Control | 4.25 | 17.25 | 29.00 | 44.47 | 57.88 | 71.29 | 84.70 | 98.11 | 111.52 | 120.46 |
| 0.008% | 2 | 0.75 | 1 | 5 | 9.25 | 13.75 | 18.25 | 21.25 | 22.75 | 24.25 |
| 0.016% | 0.75 | 0.75 | 1.50 | 6 | 10.75 | 14.50 | 18.25 | 21.00 | 21.50 | 24.25 |
| 0.032% | 1.75 | 2.00 | 1.50 | 3 | 5 | 6.50 | 8.50 | 10.75 | 12.00 | 13.25 |
| 0.064% | 3 | 2.25 | 3.00 | 4.5 | 6.25 | 7.75 | 8.75 | 11.00 | 12.25 | 12.50 |
| 0.128% | 2 | 1.75 | 2.50 | 3.25 | 5.5 | 5.75 | 8.00 | 9.75 | 11.00 | 13.00 |

### EXAMPLE 5

### Control of P. infestans (Late Blight) with a Crude Fruit Extract

Dried ivy fruit meal (94 g) was extracted with EtOH (99%, 3×250 mL) with stirring. The extract was washed with petrol (×3), dried and concentrated to provide 21 g of a solid material which appeared to be predominantly saponin-containing compounds by ¹H and ¹³C NMR spectroscopy.

The activity of the crude, defatted fruit extract against Phytophthora infestans was studied using a detached leaf assay. Crude extract (1.25 g) was dissolved in EtOH (10 ml) with gentle heating then made up with de-ionised water to 62.50 ml. A dilution series from the 2% solution was prepared of 0.200%, 0.020% and 0.002% concentrations. Leaves were removed from the stems of Solanum tuberosum var. Bintje, a Dutch potato susceptible to blight. The leaves were washed then air-dried following which they were treated with the concentration of the extract to the point of run-off then dried. The leaves were placed with the lower surface uppermost in a tray lined with moistened tissue. An aqueous suspension of sporangia (1 ml) having a concentration of 20 sporangia/μl was applied to the centre of each leaf and the tray covered. Three leaves per treatment were used and a control of ethanol (10 ml) made up to 62.5 ml with de-ionised water was included. The trial ran for twelve days by which stage the leaves had become necrotic with secondary infections.

The results after eight days following inoculation showed clearly that the control leaves were infected with lesions of P. infestans. The lowest concentration (0.002%) had one leaf infected with lesions, two others were uninfected. In all other cases, no infection of the leaves was observed. The trial continued up to twelve days showing no further infection.

### EXAMPLE 6

In an independent field trial the crude fruit extract was applied at concentrations (w/v) of 0.1% and 0.01% in water (3 L) plus an adhesion agent, (sold under the trade mark Bond) at 0.14% for each treatment concentration. Plots (four replicates of 0.08 hectares each per treatment) were planted at density of 5 plants per linear metre×4 rows per plot on May. 9, 2007.

Two control plots were included in the trial: an untreated control and a control treated with a commercial regime of different, proprietary fungicides applied thus to prevent development of resistance—the details of the commercial regime are shown in table 3.

TABLE 3
Application

| Treatment | rate Quantity |
|---|---|
| Shirlan | 0.4 l/ha. |
| Curzate M | 2 kg/ha. |
| Invader | 2 kg/ha. |
| Ranman A | 0.2 l/ha. |

Shirlan (RTM) is a suspension concentrate containing 500 g/l (38.4% w/w) fluazinam.
Curzate M (RTM) is a mixture of Mancozeb (68%), Cymoxanil(4.5%) and Hexamethylenetetramine (3.4%).
Invader (RTM) contains 75 g/kg dimethomorph and 667 g/kg mancozeb.
Ranman A (RTM) is a suspension concentrate (SC) containing 400 g/l Cyazofamid.

Treatments were applied at seven day intervals from Jun. 27, 2007 until Jul. 16, 2007 then decreasing to five day intervals during the highest blight pressure of July and August until Aug. 8, 2007 (n=9 applications). The application rate of the extracts was 300 g/hectare and 30 g/hectare respectively; the sprayer volume rate was 300 L/hectare. The crop was inoculated on Jul. 6, 2007 with a suspension of P. infestans sporangia sprayed onto 2 m wide infection strips running between the trial plots. The first infection in the untreated plots was found on Jul. 12, 2007. Following the final treatment, when conditions were no longer conducive to further infestation, the crops were left to develop, sprayed with a desiccant and finally lifted for tuber assessment on Oct. 11, 2007.

The development of leaf lesions by P. infestans was strongly inhibited by the commercial regime; control (no treatment) plots were completely destroyed by the infection. The crude fruit extract applied at 0.1% showed comparable activity to the commercial regime up to the middle of July then continued to provide significant protection until the end of treatments. This was not so with the extract at a 0.01% rate although some activity was observed relative to the untreated control. FIG. 2 shows lesion development of P. infestans on trial plots (n=4 plots per treatment).

Examination of the data that followed desiccation of the haulms and harvesting of the tubers shows that the commercial regime and the 0.01% crude extract were largely successful in preventing tuber blight; in the case of the 0.1% extract, this gave total protection. Extrapolation of the data shown in FIG. 3 showed that the number of tubers infected by blight per tonne was zero in the case of the 0.1% crude extract and outperformed even the control regime. Even at 0.01%, very high beneficial efficacy was recorded relative to the untreated plots.

The number of tubers expressed per tonne per hectare (tph) shows in FIG. 4 that the 0.1% crude extract has activity comparable to that of the control regime. When crude fruit extract is present at 0.01%, there is little infection of the tubers.

These results show clearly that there is a very significant effect by the crude extract formulations and they exhibit a strong dose response between them. Their action may have been partly due to the adhesion agent; however, this was used in both formulations at 0.14%. It was also used (at 0.1%) on a further 16 plots that were run concurrently with these trials examining other, unrelated formulations. In all cases, plots were decimated by P. infestans and no dose response was seen between them. It was also noted, that natural P. infestans, in addition to the inoculum, was high throughout the trial period at the site; rainfall was reported for the site as 462.6 mm against a thirty year average of 179.9 mm. The 2007 season was one of the wettest in many years and as such the combination of the prevalence of P. infestans' sporangia, high humidity, temperature and constant, inclement weather were ideal conditions for infestation over the trial period and meant failure for many potato growers.

FIGS. 5a to d shows pictures of the plots towards the end of the trial.

FIG. 5a shows a plot I treated with a composition comprising 0.1 wt % of a saponin component obtained according to Example 2 and an adjacent infection strip;

FIG. 5b shows plot I more closely—it can clearly be seen that the plants are in a condition similar to those treated with a commercial regime;

FIG. 5c shows a plot II treated with a composition comprising 0.1 wt % of a saponin component obtained according to Example 2; and

FIG. 5d shows plot I, an adjacent infection strip and a control plot treated with a currently used commercial formulation for combating late potato blight.

### EXAMPLE 7

### Molluscicidal/Molluscal Repellence

The extract was used in two pragmatic trials to determine the effect, if any, on the feeding behaviour of slugs in the laboratory and in a limited operational field trial.

### Laboratory Trial

A portion of the extract obtained in example 5 (0.25 g) was dissolved in MeOH (2 ml) with gentle heating (hot air gun) then made up to a 0.025% solution using H₂O (1.00 L). The solution had an opaque appearance. The solution was poured into a 1.5 L hand-held garden sprayer (Florabest, Lidl) and pressurised. Into two beakers (500 ml) were placed cabbage leaves, their bases held in aluminium foil containing wet cotton wool to retain moisture. The leaves in one beaker were then treated with a fine spray of the solution to the point of run-off. To each beaker were then added four, freshly-collected, live slugs. The beakers were covered and sealed with pierced aluminium foil and left for 48 hours.

On examination, the slugs in the treated beaker had eaten some small holes in the leaves and there were few faecal deposits. In the untreated beaker however, the leaves (with the exception of the leaf veins), were mostly eaten and a large amount of faeces was on the remains of the leaves and the base of the beaker. The slugs were subsequently released without evidence of morbidity.

### Initial Field Trial

The field trial used the same quantity of crude saponin as above that was freshly made into an aqueous solution as required. Seedlings (3 weeks old) of Brassica oleracea var. gongylodes L. (Kohl rabi) growing in pots in a greenhouse were treated with the solution by spraying, to the point of run-off, in late April, 2005. A control sample was left untreated on another propagation table. The treatments continued bi-weekly until both sets of seedlings were planted out in mid-May. The treated seedlings then continued to receive treatment weekly until mid-June. The results (FIGS. 6a and 6b) demonstrated that an effect between the treated and untreated seedlings had occurred with more vigorous growth and less damage by browsing to the leaves of the treated sample.

FIG. 6a shows the untreated plants and FIG. 6b shows the treated plants.

### Controlled Slug Trials 1

Anion ater and A. rufus species were collected at Treborth Botanical Gardens from under logs. Prior to the trial they were starved for 24 hours. 10 random slugs were introduced to each of 13 trays. The trays contained 6 plants each (Tagetes patula, French marigold) and were subjected to 4 treatments. Concentrations were low (0.025 wt %), medium (0.050 wt %) and high (0.500 wt %) applied as a spray to the point of run off. Primary (24 hr) & residual (48 hr) effects were recorded by counting the number of slugs on the plants. The results are shown in FIG. 7.

### Controlled Slug Trials 2

To obtain quantitative data of damage, leaf discs (6.5 cm) were cut from Brassica oleracea, var. acephala (collards/spring greens). Discs were sprayed as described in the first trial to run off using the same concentrations and a further ultra low concentration at 0.010% then placed in a marked Petri dish. The position of the treated leaves in the trays was statistically randomised. Anion ater and Anion rufus were the species of slug used. A 24 hours starvation period preceded the trial. 4 slugs were introduced per tray containing the treated discs. After 24 hours, the leaf discs were photographed individually, from the same distance, and then the digital images were processed in Microsoft Powerpoint to produce a black image. This was printed on to acetate film and each disc was cut out. Using a simple leaf area index apparatus (Li-Cor model TT3050A/4), data was obtained of the area of leaf consumed. The results are presented in FIG. 8. FIGS. 9a, 9b and 9c shows the effect on trays 1, 3 and 5 respectively.

### Controlled Slug Trials 3

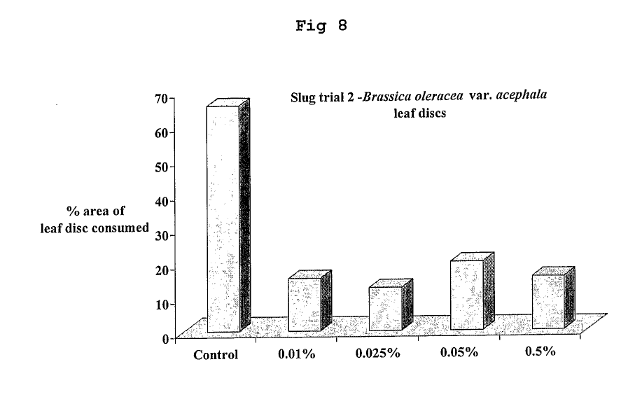

A further trial was repeated using A. ater and A. rufus. To minimise the effects of leaf morphology, following the trial, the leaves were digitally scanned and then passed through the Li-Cor leaf area index apparatus. These results are shown in FIG. 10.

### EXAMPLE 8

### Base Hydrolysis of Saponin-Containing Component

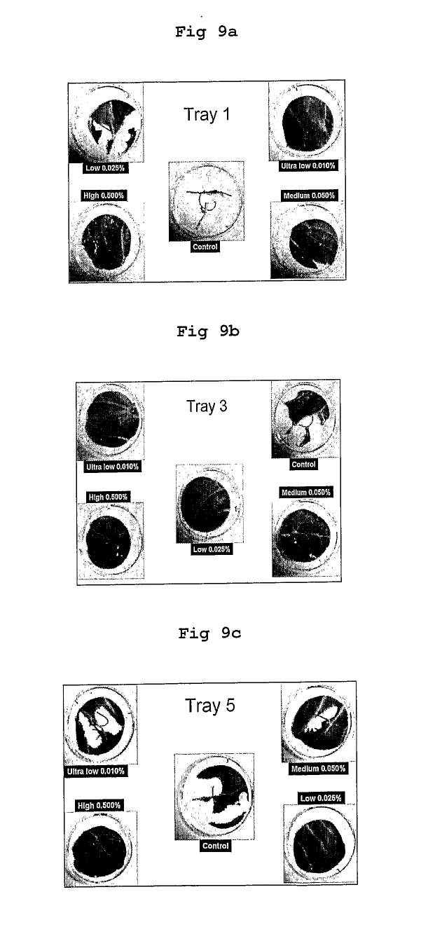

A sample of substantially de-fatted crude saponins from H. helix fruits (10 g) was reacted for 7 hours at 70° C. (oil bath) in a solution of NaOH (40 ml, 2M). The mixture, as a slurry, was transferred to a separating funnel and a solution of NaOH (20 ml, 2M) used to wash the flask. The solution was acidified to pH 2 with a 10% (v/v) solution of H₂SO₄ (60 ml) whereupon a precipitate formed. The product was taken up in n-BuOH and the aqueous layer drawn off. The solvent was removed in vacuo to recover a solid (7.54 g). The ¹H-NMR spectrum is shown in FIG. 11.

### EXAMPLE 9

### Acid Hydrolysis of Saponin-Containing Component

Crude fruit extract (40.00 g) was stirred under reflux with 10% HCl (250 ml) and MeOH (20 ml) at 70° C. (oil bath) overnight. A solid was present throughout the reaction as the major component. The reaction mixture was cooled, filtered through a Buchner funnel under reduced pressure and the solid dried overnight in an oven at 50° C. to afford a dark brown, greasy slurry. This was washed with Et₂O (200 ml) from which was obtained a small residue after filtration and evaporation. The remainder was taken up in n-BuOH (500 ml) and filtered from which a further small residue was obtained from the filter paper. The n-BuOH was removed in vacuo to recover a grey solid that was then washed with EtOAc (200 ml), dried to a cake then crushed in a pestle and mortar to give a fine grey powder (12.81 g). A sample of this was submitted for ¹H- and ¹³C-NMR analysis. The ¹H-NMR spectrum showed a product clear of sugar signals that essentially matched the standard of hederagenin (Extrasynthese, France). The chemical shifts in ¹³C-NMR matched those of both literature and that of the standard. Both are detailed in table 4.

TABLE 4

| Shift, ppm | Shift, ppm |
|---|---|
| reaction | literature (and |
| product in | standard) in |
| CD₃OD | C₅D₅N |
| 12.72 | 13.02 (13.13) |
| 16.28 | 15.94 (15.96) |
| 17.76 | 17.46 (17.49) |
| 19.16 | 18.58 (15.58) |
| 24.00 | 23.68 (23.69) |
| 24.06 | 23.77 (23.75) |
| 24.52 | 23.82 (23.84) |
| 26.47 | 26.15 (26.14) |
| 27.41 | 27.54 (27.68) |
| 28.84 | 28.30 (28.33) |
| 31.61 | 30.92 (30.94) |
| 33.49 | 32.95 (32.97) |
| 33.58 | 33.18 (33.20) |
| 33.81 | 33.24 (33.23) |
| 34.91 | 34.22 (34.20) |
| 37.92 | 37.78 (37.23) |
| 39.49 | 38.9 (38.78) |
| 40.51 | 39.75 (39.76) |
| 42.73 | 41.98 (41.99) |
| 42.97 | 42.18 (42.18) |
| 43.26 | 42.81 (42.88) |
| 47.24 | 46.47 (46.44) |
| 47.62 | 46.65 (46.65) |
| 49.12 | 48.13 (48.15) |
| 49.28 | 48.65 (48.60) |
| 67.49 | 68.01 (67.89) |
| 73.99 | 73.52 (73.37) |
| 123.60 | 122.55 (122.58) |
| 145.24 | 144.81 (144.84) |
| 181.81 | 180.17 (180.21) |

### EXAMPLE 10

### Independent Trials of Slug Activity with a Crude Saponin Extract—Preliminary Results

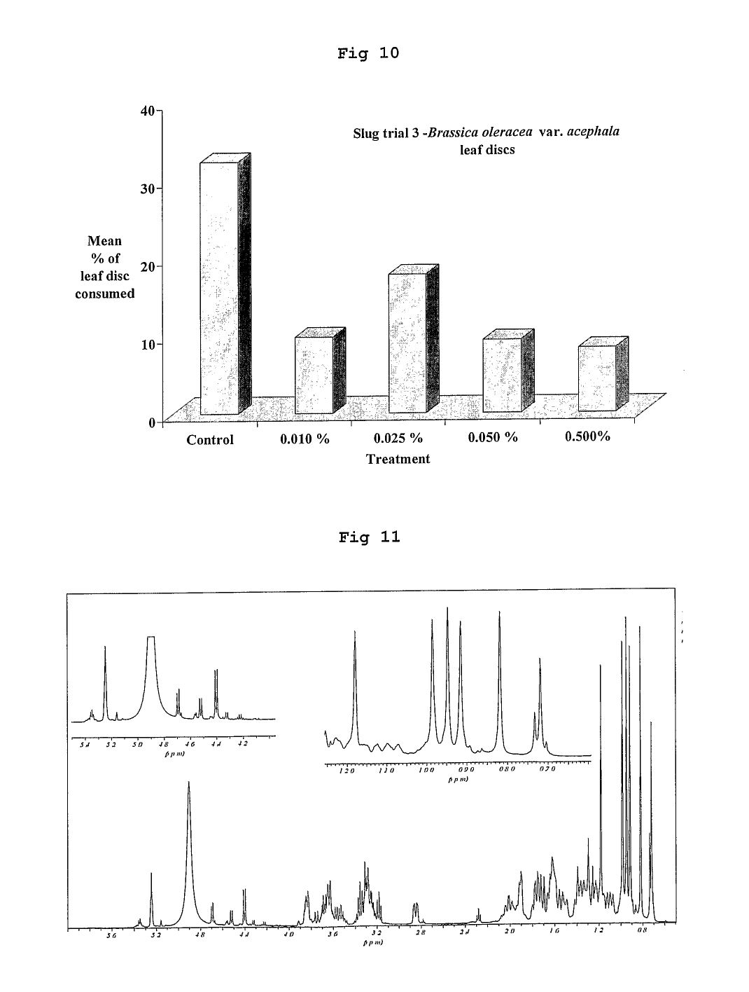

In an independent assessment, the percentage of leaf disc consumed following exposure of Dereocereus reticulatum to crude extracts of leaves of Hedera Helix, crude extracts of fruits of Hedera Helix, and a base hydrolysis product of the fruit extract were measured. In each case solutions comprising 0.1 wt %, 0.01 wt % and 0.001 wt % of the extract was applied. The results are of FIG. 12a show the effects on browsing of slugs and those of FIG. 12b show the effects on mortality.

### EXAMPLE 11

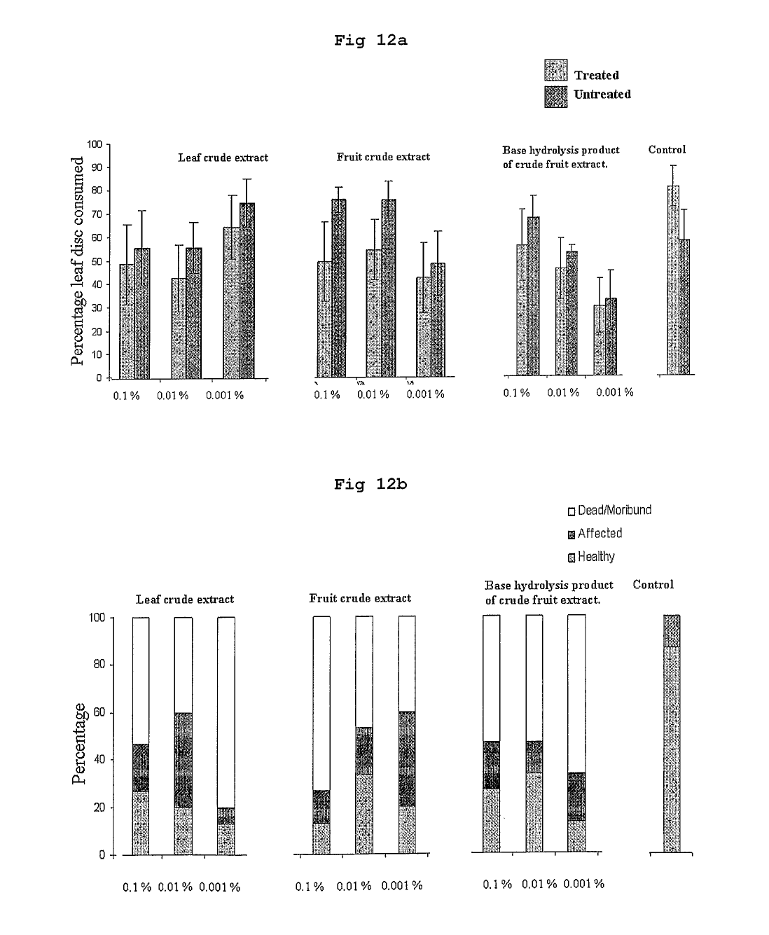

5 g of crude fatty acid residue obtained from the seeds of Hedera Helix were dissolved in 10 mL acetone and cooled to 1.1° C. for 48 hours. The yellow crystals that formed were collected and recrystallised from acetone to provide 2.66 g of white crystals. The ¹³C NMR spectrum of the material shown in FIG. 13 indicated that the crystals were predominantly tripetroselinin.

### EXAMPLE 12

The following compositions were tested for activity against the fungal species Candida albicans:

A—a composition comprising the crude fruit extract obtained in example 2;
B—a composition comprising the base hydrolysis product of example 9;
C—a composition comprising the acid hydrolysis product of example 8; and
D—a composition comprising a base hydrolysis product of a crude saponin-containing component extracted from leaves of Hedera Helix.

In each case, compositions A to D were dissolved in DMSO and then diluted in the assay culture media to give a range of concentrations. To each sample a composition comprising a culture of Candida albicans was added and the minimum concentration of test component needed to inhibit growth of the culture was determined, compared to a drug-free control.

Although samples A and B were found to be effective at inhibiting the growth of Candida albicans, sample D was found to be particularly effective and inhibited 100% of the growth of Candida albicans, at a concentration of 8 mgdm⁻³.

### EXAMPLE 13

When sample D of example 12 was tested against Aspergillus fumigatus, it was found to inhibit 50% of the growth at a concentration of 64 mgdm⁻³.

### EXAMPLE 14

Sample C was found to have a dose-dependent effect on controlling the growth of Staphylococcus aureus.

### EXAMPLE 15

The effect on the mortality of potato cyst nematode (PCN) following treatment with varying concentrations of the compositions A to D in example 12 was measured.

Hatched juveniles were exposed to compositions comprising varying concentrations in water. The results in table 5 show the percentage mortality in PLN juveniles exposed to the test solutions.

The mortality % is calculated from 4 replicates of each concentration, counted 3 times.

TABLE 5

| | Treatment | | | |
|---|---|---|---|---|
| | Rate | Hours | | |
| | (% w/v) | 1 | 3 | 5 |
| Control | | 10.2 | 6.1 | 5.4 |
| A | 0.01 | 27.3 | 29 | 20.5 |
| | 0.001 | 25 | 20 | 20 |
| | 0.0001 | 12.4 | 15.4 | 13.2 |
| B | 0.01 | 33.3 | 51.7 | 52.8 |
| | 0.001 | 19.4 | 34.2 | 44.8 |
| | 0.0001 | 18 | 27.3 | 30.4 |
| C | 0.01 | 23.1 | 33.3 | 35.5 |
| | 0.001 | 19.4 | 23.1 | 23.3 |
| | 0.0001 | 21.1 | 27.8 | 25 |
| D | 0.01 | 17.5 | 21.6 | 25 |
| | 0.001 | 10.7 | 17.1 | 11.8 |
| | 0.0001 | 12.5 | 18.9 | 13.3 |

### EXAMPLE 16

Potato plants were grown in field plots where populations of field slugs (Derocerus reticulatum, Anion. spp. and keeled slugs) were present. Crude H. helix fruit extract obtained by example 2 was applied to the soil by spraying at an application rate of 600 g/ha with a sprayer volume of 300 L/ha. In addition to a control treatment, applications by a knapsack sprayer were made at either 2 weekly or four weekly intervals over the period of tuber development. Following post-harvest examination for slug damage, a positive effect in controlling slug damage to the tubers was established for the crude fruit extract when compared to the control treatment.

### EXAMPLE 17

An accelerated decay test was conducted using a widely accepted method, presenting treated and untreated blocks to cultures of pure fungus on agar. Three brown rot test fungi were used
Coniophora puteana, Serpula lacrymans and Poria placenta. These are common in the decay of timber in buildings.

Scots pine miniblocks (5×10×30 mm) were impregnated with a crude saponin-containing component obtained from leaves of Hedera Helix; a crude saponin containing components obtained from the fruit of Hedera Helix; and the base hydrolysis product of the fruit extract. The miniblocks were arranged in the petri dishes so that three treated and three untreated blocks alternated around the dish.

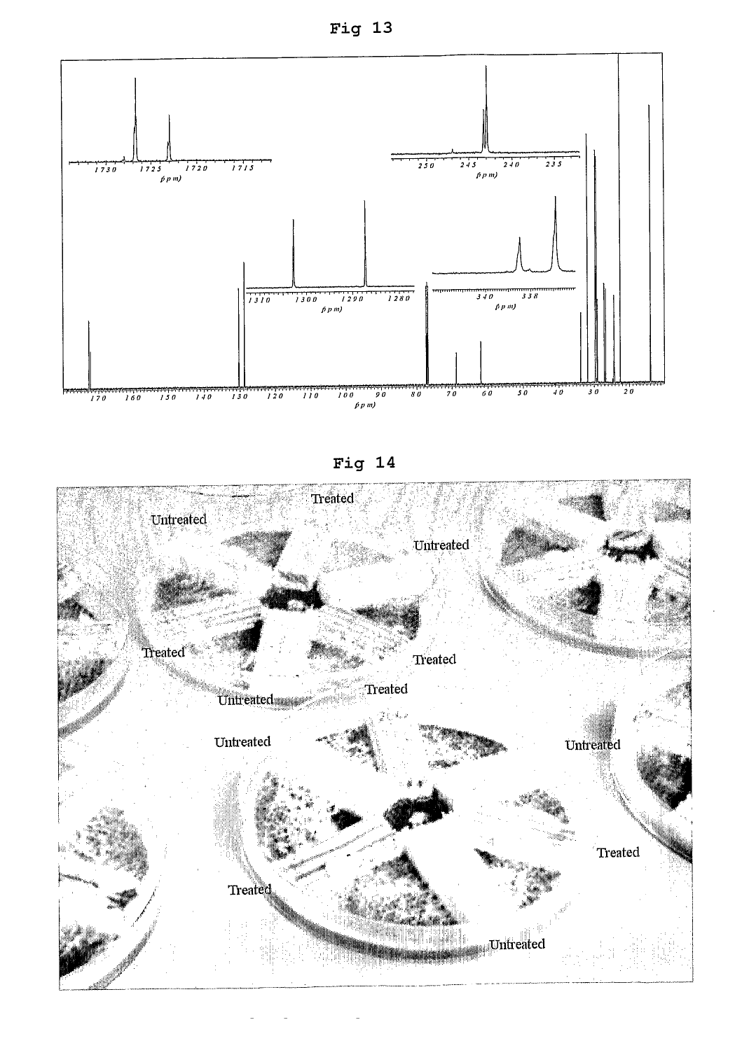

The blocks treated with all three extracts showed a decrease in weight loss after exposure to the three brown rot fungi. The weight loss was suppressed to negligible levels in the blocks exposed to Poria placenta, and the difference in colonisation by the mycelium was clearly visible, as shown in FIG. 14.

1. A process of obtaining a saponin-rich component from a plant of the Araliaceae family, the process comprising the steps of:
(a) treating a portion of the plant with an extraction solvent in which saponin-containing compounds are soluble; and
(b) treating the portion of plant or the extract obtained therefrom to remove fatty acid residues from said portion of plant or extract thereof.

2. A process according to claim 1 wherein the portion of the plant comprises the fruit of the plant.

3. A process according to claim 1 wherein the plant comprises Hedera Helix.

4. A process according to claim 1 wherein the extraction solvent comprises an alcohol.

5. A process according to claim 1 in which step (a) is carried out prior to step (b).

6. A process according to claim 1 wherein the fatty acid residue removed in step (b) is retained.

7. A process according to claim 6 which further comprises hydrolysing glycerol triesters to provide one or more of petroselinic acid, palmitoleic acid and vaccenic acid.

8. A process according to claim 6 which further comprises dissolving the fatty acid residue in a crystallisation solvent, cooling the solution to a temperature of less than 5° C., and collecting the solid precipitate.

9. The glycerol triester of petroselinic acid prepared by the process of claim 8.

10. A process according to claim 7 which further comprises dissolving the fatty acid hydrolysis product in a crystallisation solvent, cooling the solution to a temperature less than 5° C., and collecting the solid precipitate.

11. Petroselinic acid prepared by the process of claim 10.

12. The fatty acid residue obtained by the process of claim 6.

13-24. (canceled)

25. Compounds having the formula: 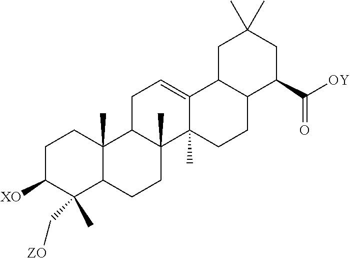
wherein X, Y and Z are independently selected from hydrogen, a fatty acid residue or a sugar residue and wherein at least one of X, Y and Z is not hydrogen.

26. Compounds having the formula: 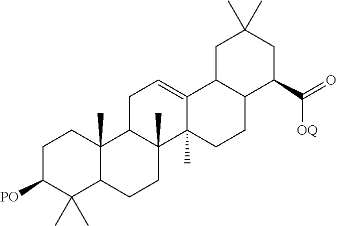
wherein P and Q can be independently selected from hydrogen, a fatty acid residue or a sugar residue and wherein at least one of P and Q is not hydrogen.

27. A composition comprising a compound as claimed in claim 25.

28. A method of manufacture of a compound as claimed in claim 25.

29. Compounds of claim 25 for use in therapy.

30. The use of a compound according to claim 25 as a molluscicide and/or a mollusc antifeedant and/or a mollusc repellent; in a method of combating potato blight; as a fungicide, a fungistatic agent or as an adjuvant in fungal control.

31. A method of manufacture of a compound as claimed in claim 26.

32. Compounds of claim 26 for use in therapy.
